(19) United States
(12) Patent Application Publication
(10) Pub. No.: US 2019/0382396 A1
(43) Pub. Date: Dec. 19, 2019

(54) SALTS OF BICYCLO[1.1.1]PENTANE INHIBITORS OF DUAL LEUCINE ZIPPER (DLK) KINASE FOR THE TREATMENT OF DISEASE

(71) Applicant: Board of Regents, The University of Texas System, Austin, TX (US)

(72) Inventors: Philip JONES, Houston, TX (US); Michael J. SOTH, Sugar Land, TX (US); Kang LE, Sugar Land, TX (US)

(21) Appl. No.: 16/437,785

(22) Filed: Jun. 11, 2019

Related U.S. Application Data
(60) Provisional application No. 62/684,574, filed on Jun. 13, 2018.

(51) Int. Cl. C07D 413/14

(52) U.S. Cl. CPC C07D 413/14

(57) ABSTRACT

Disclosed herein are new substituted bicyclo[1.1.1]pentane compounds which inhibit the kinase activity of dual leucine zipper (DLK) kinase (MAP3K12). Also disclosed herein are solid polymorph forms of these compounds. Also disclosed herein are salts of these compounds, and solid polymorph forms of these salts. Also disclosed herein are compounds, pharmaceutical compositions, and methods of treatment of DLK-mediated diseases, such as neurological diseases that result from traumatic injury to central nervous system and peripheral nervous system neurons, or that result from a chronic neurodegenerative condition, from neuropathies resulting from neurological damage and from cognitive disorders caused by pharmacological intervention.

This application claims the benefit of priority of U.S. Provisional Application No. 62/684,574, filed 13 Jun. 2018, the disclosure of which is hereby incorporated by reference as if written herein in its entirety.

Disclosed herein are new substituted bicyclo[1.1.1]pentane compounds and compositions and their application as pharmaceuticals for the treatment of disease. Also disclosed herein are solid polymorph forms of these compounds. Also disclosed herein are salts of these compounds, and solid polymorph forms of these salts. Methods of inhibition of the kinase activity of dual leucine zipper in a human or animal subject are also provided for the treatment of diseases such as neurological diseases that result from traumatic injury to central nervous system and peripheral nervous system neurons, neurodegenerative conditions, neuropathies resulting from neurological damage, and treatment of pain and cognitive disorders caused by pharmacological intervention.

Dual leucine zipper kinase (DLK) is a member of the mixed lineage kinase (MLK) family that is required for stress-induced neuronal activation of c-Jun N-terminal kinases (JNK). In turn, JNK is implicated in pathways important to cellular regulation including apoptosis and cell proliferation. JNK has been implicated in both naturally occurring cell death and pathological death of neurons.

Novel compounds and pharmaceutical compositions, certain of which have been found to inhibit the kinase activity of DLK have been discovered, together with methods of synthesizing and using the compounds including methods for the treatment of DLK-mediated diseases in a patient by administering the compounds.

### BRIEF DESCRIPTION OF THE DRAWINGS

FIG. 1 shows XRPD diffractograms of Example Compound 1 from various solvents.

FIG. 2 shows XRPD diffractograms of Example Compound 1 from various solvents.

FIG. 3 shows XRPD diffractograms of Example Compound 1 from various solvents.

FIG. 4 shows XRPD diffractograms from salt formation experiments of Example Compound 1 with tartaric acid.

FIG. 5 shows XRPD diffractograms from salt formation experiments of Example Compound 1 with L-malic acid.

FIG. 6 shows XRPD diffractograms from salt formation experiments of Example Compound 1 with citric acid.

FIG. 7 shows XRPD diffractograms from salt formation experiments of Example Compound 1 with maleic acid.

FIG. 8 shows XRPD diffractograms from salt formation experiments of Example Compound 1 with succinic acid.

FIG. 9 shows XRPD diffractograms from salt formation experiments of Example Compound 1 with fumaric acid.

FIG. 10 shows DSC of dihydrogen citrate salt 8.

FIG. 11 shows DSC of hydrogen maleate salt 9.

FIG. 12 shows DSC of hydrogen succinate salt 10.

FIG. 13 shows DSC of hydrogen fumarate salt 11.

FIG. 14 shows XRPD diffractograms of scale up of hydrogen fumarate salt 11.

FIG. 15 shows ¹H NMR hydrogen fumarate salt 11.

FIG. 16 shows XRPD diffractograms of hydrogen fumarate salt 11 before and after DVS.

FIG. 17 shows XRPD diffractograms from physical stability study for hydrogen fumarate salt 11.

FIG. 18 shows XRPD diffractograms from different batches of hydrogen maleate salt 9.

FIG. 19 shows XRPD diffractograms from different batches of hydrogen succinate salt 10.

FIG. 20 shows XRPD diffractograms from different batches of hydrogen fumarate salt 11.

FIG. 21 shows XRPD diffractograms of hydrogen fumarate salt 11 slurries from various solvents.

FIG. 22 shows XRPD diffractograms of hydrogen fumarate salt 11 slurries from various solvents.

FIG. 23 shows DSC/TGA overlay of hydrogen fumarate salt 11 from acetone.

FIG. 24 shows DSC/TGA overlay of hydrogen fumarate salt 11 from isopropanol.

FIG. 25 shows XRPD diffractograms of hydrogen fumarate salt 11 from polymorph formation experiment.

FIG. 26 shows XRPD diffractograms of hydrogen fumarate salt 11 from competitive slurry study.

FIG. 27 shows the XRPD diffractogram of hydrogen fumarate salt 11 from the scaled up synthesis.

FIG. 28 shows the ¹H NMR (DMSO-d₆) of hydrogen fumarate salt 11 from the scaled up synthesis.

FIG. 29 shows a particle size distribution of hydrogen fumarate salt 11 from the scaled up synthesis.

### DETAILED DESCRIPTION

Provided herein is Embodiment 1: a compound have structural Formula I:

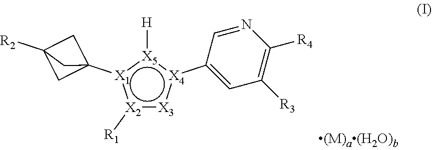

or a polymorph thereof, wherein:

X₁ is selected from C and N;

X₂ is selected from C and N;

exactly one of X₁ and X₂ is N;

X₃ is N;

X₄ and X₅ are C;

X₁, X₂, X₃, X₄, and X₅ form a five membered heteroaryl;

R₁ is selected from alkyl, cycloalkyl, and heterocycloalkyl, any of which is optionally substituted with one to three R₅ groups;

R₂ is H or is selected from alkyl, amino, aryl, cycloalkyl, haloalkyl, heteroalkyl, heteroaryl, heterocycloalkyl, and sulfonylalkyl, any of which is optionally substituted with one to three R₆ groups;

R₃ is selected from H, alkyl, (alkoxy)alkyl, (arylalkoxy)alkyl, (heteroarylalkoxy)alkyl, cyano, cycloalkyl, halo, haloalkoxy, and haloalkyl;

R₄ is N(R₄ₐ)₂, wherein each R₄ₐ is independently selected from hydrogen, C₁₋₄alkyl, and C₁₋₄haloalkyl;

or R₃ and R₄ together with the atoms to which they are attached form a 5- or 6-membered heteroaryl or heteroalkyl ring, optionally substituted with one to three R₇ groups;

each R₅ and R₆ is independently selected from C₁₋₄alkyl, C₁₋₄haloalkyl, C₁₋₄alkoxy, C₁₋₄haloalkoxy, C₁₋₄alkylthio, C₁₋₄haloalkylthio, aryl, heteroaryl, C₃₋₇cycloalkyl, C₃₋₇heterocycloalkyl, (aryl)C₁₋₄alkyl, (heteroaryl)C₁₋₄alkyl, (C₃₋₇cycloalkyl)C₁₋₄alkyl, (C₃₋₇heterocycloalkyl)C₁₋₄alkyl, (ethenyl)C₁₋₄alkyl, (ethynyl)C₁₋₄alkyl, (aryl)C₁₋₄alkoxy, (heteroaryl)C₁₋₄alkoxy, (C₃₋₇cycloalkyl)C₁₋₄alkoxy, (C₃₋₇heterocycloalkyl)C₁₋₄alkoxy, (aryl)C₁-4alkylthio, (heteroaryl)C₁₋₄alkylthio, (C₃₋₇cycloalkyl)C₁₋₄alkylthio, (C₃₋₇heterocycloalkyl)C₁-4alkylthio, amino, halo, hydroxy, cyano, and oxo;

each R₇ is independently selected from C₁₋₄alkyl, C₁₋₄haloalkyl, C₁₋₄alkoxy, C₁₋₄haloalkoxy, aryl, heteroaryl, C₃₋₇cycloalkyl, C₃₋₇heterocycloalkyl, (aryl)C₁₋₄alkyl, (heteroaryl)C₁₋₄alkyl, (C₃₋₇cycloalkyl)C₁₋₄alkyl, (C₃₋₇heterocycloalkyl)C₁₋₄alkyl, halo, hydroxy, cyano, and oxo;

a is a fractional or whole number between about 0.5 and 3.5, inclusive;

b is a fractional or whole number between about 0 and 10, inclusive; and

M is selected from hydrochloric acid, sulfuric acid, phosphoric acid, maleic acid, fumaric acid, tartaric acid, succinic acid, L-malic acid, citric acid, and methanesulfonic acid.

Certain compounds disclosed herein possess useful DLK inhibiting activity, and may be used in the treatment or prophylaxis of a disease in which DLK plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting DLK. Other embodiments provide methods for treating a DLK-mediated disorder in a patient in need of such treatment, comprising administering to said patient a therapeutically effective amount of a compound or composition as disclosed herein. Also provided is the use of certain compounds disclosed herein for use in the manufacture of a medicament for the treatment of a disease ameliorated by the inhibition of DLK.

In certain embodiments, M is chosen from maleic acid, fumaric acid, succinic acid, and citric acid.

The disclosure provides the further embodiments:

### Embodiment 2

The compound of Embodiment 1, wherein a is a fractional or whole number between about 0.5 and 3.0, inclusive.

### Embodiment 3

The compound of Embodiment 2, wherein a is a fractional or whole number between about 0.5 and 2.5, inclusive.

### Embodiment 4

The compound of Embodiment 3, wherein a is a fractional or whole number between about 0.5 and 2.0, inclusive.

### Embodiment 5

The compound of Embodiment 4, wherein a is a fractional or whole number between about 0.5 and 1.5, inclusive.

### Embodiment 6

The compound of Embodiment 5, wherein a is a fractional or whole number between about 0.5 and 1.0, inclusive.

### Embodiment 7

The compound of Embodiment 1, wherein a is a fractional or whole number between about 1.0 and 2.0, inclusive.

### Embodiment 8

The compound of Embodiment 1, wherein a is a fractional or whole number between about 2.0 and 3.0, inclusive.

### Embodiment 9

The compound of Embodiment 1, wherein a is about 1.0.

### Embodiment 10

The compound of Embodiment 1, wherein a is 1.0.

### Embodiment 11

The compound of Embodiment 1, wherein a is about 1.5.

### Embodiment 12

The compound of Embodiment 1, wherein a is 1.5.

### Embodiment 13

The compound of Embodiment 1, wherein a is about 2.0.

### Embodiment 14

The compound of Embodiment 1, wherein a is 2.0.

### Embodiment 15

The compound of Embodiment 1, wherein a is about 3.0.

### Embodiment 16

The compound of Embodiment 1, wherein a is 3.0.

### Embodiment 17

The compound of any one of Embodiments 1 through 16, wherein R₁ is isopropyl.

### Embodiment 18

The compound of any one of Embodiments 1 through 17, wherein R₂ is morpholin-1-yl.

### Embodiment 19

The compound of any one of Embodiments 1 through 18, wherein wherein R₃ is selected from difluoromethoxy, trifluoromethoxy, and trifluoromethyl.

### Embodiment 20

The compound of Embodiment 19, wherein R₃ is trifluoromethoxy.

### Embodiment 21

The compound of any one of Embodiments 1 through 20, wherein R₄ is NH₂.

Also provided herein is Embodiment 22: a compound of Formula II:

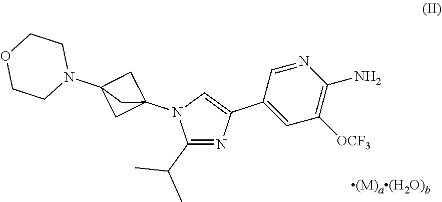 [SMILES: CC(C)c1nc(-c2cnc(N)c(OC(F)(F)F)c2)cn1C12CC(N3CCOCC3)(C1)C2]

or a polymorph thereof, wherein:

a is a fractional or whole number between about 0.5 and 1.5, inclusive;

b is a fractional or whole number between about 0 and 10, inclusive; and

M is selected from an organic acid, an inorganic acid, and an amino acid.

The disclosure provides the further embodiments:

### Embodiment 23

The compound of any one of Embodiments 1 through 22, wherein the compound is in a solid form.

### Embodiment 24

The compound of Embodiment 23, wherein the compound is in a crystalline form.

### Embodiment 25

The compound of any one of Embodiments 1 through 24, wherein M is chosen from HCl, H₂SO₄, H₃PO₄, methanesulfonic acid, tartaric acid, L-malic acid, citric acid, maleic acid, succinic acid, and fumaric acid.

### Embodiment 26

The compound of Embodiment 25, wherein M is chosen from tartaric acid, L-malic acid, citric acid, maleic acid, succinic acid, and fumaric acid.

### Embodiment 27

The compound of Embodiment 26, wherein M is chosen from citric acid, maleic acid, succinic acid, and fumaric acid.

### Embodiment 28

The compound of Embodiment 27, wherein M is chosen from maleic acid, succinic acid, and fumaric acid.

### Embodiment 29

The compound of Embodiment 28, wherein M is citric acid.

### Embodiment 30

The compound of Embodiment 28, wherein M is maleic acid.

### Embodiment 31

The compound of Embodiment 28, wherein M is succinic acid.

### Embodiment 32

The compound of Embodiment 28, wherein M is fumaric acid.

### Embodiment 33

The compound of Embodiment 10, chosen from:
5-(2-isopropyl-1-(3-morpholinobicyclo[1.1.1]pentan-1-yl)-1H-imidazol-4-yl)-3-(trifluoromethoxy)pyridin-2-amine hydrochloride;
5-(2-isopropyl-1-(3-morpholinobicyclo[1.1.1]pentan-1-yl)-1H-imidazol-4-yl)-3-(trifluoromethoxy)pyridin-2-amine hydrogen sulfate;
5-(2-isopropyl-1-(3-morpholinobicyclo[1.1.1]pentan-1-yl)-1H-imidazol-4-yl)-3-(trifluoromethoxy)pyridin-2-amine dihydrogen phosphate;
5-(2-isopropyl-1-(3-morpholinobicyclo[1.1.1]pentan-1-yl)-1H-imidazol-4-yl)-3-(trifluoromethoxy)pyridin-2-amine methanesulfonate;
5-(2-isopropyl-1-(3-morpholinobicyclo[1.1.1]pentan-1-yl)-1H-imidazol-4-yl)-3-(trifluoromethoxy)pyridin-2-amine hydrogen tartrate;
5-(2-isopropyl-1-(3-morpholinobicyclo[1.1.1]pentan-1-yl)-1H-imidazol-4-yl)-3-(trifluoromethoxy)pyridin-2-amine hydrogen L-malate;
5-(2-isopropyl-1-(3-morpholinobicyclo[1.1.1]pentan-1-yl)-1H-imidazol-4-yl)-3-(trifluoromethoxy)pyridin-2-amine dihydrogen citrate;
5-(2-isopropyl-1-(3-morpholinobicyclo[1.1.1]pentan-1-yl)-1H-imidazol-4-yl)-3-(trifluoromethoxy)pyridin-2-amine hydrogen maleate;
5-(2-isopropyl-1-(3-morpholinobicyclo[1.1.1]pentan-1-yl)-1H-imidazol-4-yl)-3-(trifluoromethoxy)pyridin-2-amine hydrogen succinate; and
5-(2-isopropyl-1-(3-morpholinobicyclo[1.1.1]pentan-1-yl)-1H-imidazol-4-yl)-3-(trifluoromethoxy)pyridin-2-amine hydrogen fumarate.

### Embodiment 34

The compound of Embodiment 10, chosen from:
5-(2-isopropyl-1-(3-morpholinobicyclo[1.1.1]pentan-1-yl)-1H-imidazol-4-yl)-3-(trifluoromethoxy)pyridin-2-amine hydrogen tartrate;
5-(2-isopropyl-1-(3-morpholinobicyclo[1.1.1]pentan-1-yl)-1H-imidazol-4-yl)-3-(trifluoromethoxy)pyridin-2-amine hydrogen L-malate;
5-(2-isopropyl-1-(3-morpholinobicyclo[1.1.1]pentan-1-yl)-1H-imidazol-4-yl)-3-(trifluoromethoxy)pyridin-2-amine dihydrogen citrate;
5-(2-isopropyl-1-(3-morpholinobicyclo[1.1.1]pentan-1-yl)-1H-imidazol-4-yl)-3-(trifluoromethoxy)pyridin-2-amine hydrogen maleate;
5-(2-isopropyl-1-(3-morpholinobicyclo[1.1.1]pentan-1-yl)-1H-imidazol-4-yl)-3-(trifluoromethoxy)pyridin-2-amine hydrogen succinate; and
5-(2-isopropyl-1-(3-morpholinobicyclo[1.1.1]pentan-1-yl)-1H-imidazol-4-yl)-3-(trifluoromethoxy)pyridin-2-amine hydrogen fumarate.

### Embodiment 35

The compound of Embodiment 10, chosen from:
5-(2-isopropyl-1-(3-morpholinobicyclo[1.1.1]pentan-1-yl)-1H-imidazol-4-yl)-3-(trifluoromethoxy)pyridin-2-amine dihydrogen citrate;
5-(2-isopropyl-1-(3-morpholinobicyclo[1.1.1]pentan-1-yl)-1H-imidazol-4-yl)-3-(trifluoromethoxy)pyridin-2-amine hydrogen maleate;
5-(2-isopropyl-1-(3-morpholinobicyclo[1.1.1]pentan-1-yl)-1H-imidazol-4-yl)-3-(trifluoromethoxy)pyridin-2-amine hydrogen succinate; and
5-(2-isopropyl-1-(3-morpholinobicyclo[1.1.1]pentan-1-yl)-1H-imidazol-4-yl)-3-(trifluoromethoxy)pyridin-2-amine hydrogen fumarate.

### Embodiment 36

The compound of Embodiment 10, chosen from:
5-(2-isopropyl-1-(3-morpholinobicyclo[1.1.1]pentan-1-yl)-1H-imidazol-4-yl)-3-(trifluoromethoxy)pyridin-2-amine hydrogen maleate;
5-(2-isopropyl-1-(3-morpholinobicyclo[1.1.1]pentan-1-yl)-1H-imidazol-4-yl)-3-(trifluoromethoxy)pyridin-2-amine hydrogen succinate; and
5-(2-isopropyl-1-(3-morpholinobicyclo[1.1.1]pentan-1-yl)-1H-imidazol-4-yl)-3-(trifluoromethoxy)pyridin-2-amine hydrogen fumarate.

### Embodiment 37

The compound of Embodiment 10, wherein the compound is 5-(2-isopropyl-1-(3-morpholinobicyclo[1.1.1]pentan-1-yl)-1H-imidazol-4-yl)-3-(trifluoromethoxy)-pyridin-2-amine dihydrogen citrate.

### Embodiment 38

The compound of Embodiment 37, characterized by the presence of four or more XRPD peaks with d-spacings of about 26.87, 24.26, 6.62, 5.86, 5.15, 4.68, 4.60, 4.52, 4.38, and 4.19 Å.

### Embodiment 39

The compound of Embodiment 38, characterized by the presence of five or more XRPD peaks with d-spacings of about 26.87, 24.26, 6.62, 5.86, 5.15, 4.68, 4.60, 4.52, 4.38, and 4.19 Å.

### Embodiment 40

The compound of Embodiment 39, characterized by the presence of six or more XRPD peaks with d-spacings of about 26.87, 24.26, 6.62, 5.86, 5.15, 4.68, 4.60, 4.52, 4.38, and 4.19 Å.

### Embodiment 41

The compound of Embodiment 40, characterized by the presence of seven or more XRPD peaks with d-spacings of about 26.87, 24.26, 6.62, 5.86, 5.15, 4.68, 4.60, 4.52, 4.38, and 4.19 Å.

### Embodiment 42

The compound of Embodiment 41, characterized by the presence of eight or more XRPD peaks with d-spacings of about 26.87, 24.26, 6.62, 5.86, 5.15, 4.68, 4.60, 4.52, 4.38, and 4.19 Å.

### Embodiment 43

The compound of Embodiment 37, characterized by the presence of four or more XRPD peaks with 2-theta values, using CuK radiation, of about 3.29, 3.64, 13.36, 15.11, 17.22, 18.94, 19.27, 19.61, 20.26, and 21.21 degrees.

### Embodiment 44

The compound of Embodiment 43, characterized by the presence of five or more XRPD peaks with 2-theta values, using CuK radiation, of about 3.29, 3.64, 13.36, 15.11, 17.22, 18.94, 19.27, 19.61, 20.26, and 21.21 degrees.

### Embodiment 45

The compound of Embodiment 44, characterized by the presence of six or more XRPD peaks with 2-theta values, using CuK radiation, of about 3.29, 3.64, 13.36, 15.11, 17.22, 18.94, 19.27, 19.61, 20.26, and 21.21 degrees.

### Embodiment 46

The compound of Embodiment 45, characterized by the presence of seven or more XRPD peaks with 2-theta values, using CuK radiation, of about 3.29, 3.64, 13.36, 15.11, 17.22, 18.94, 19.27, 19.61, 20.26, and 21.21 degrees.

### Embodiment 47

The compound of Embodiment 46, characterized by the presence of eight or more XRPD peaks with 2-theta values, using CuK radiation, of about 3.29, 3.64, 13.36, 15.11, 17.22, 18.94, 19.27, 19.61, 20.26, and 21.21 degrees.

### Embodiment 48

The compound of any one of Embodiments 37 through 47, characterized by the presence of an exothermic peak in the DSC at about 175° C.

### Embodiment 49

The compound of any one of Embodiments 37 through 47, characterized by a weight loss of no more than 0.2% below 120° C. by TGA.

### Embodiment 50

The compound of Embodiment 10, wherein the compound is 5-(2-isopropyl-1-(3-morpholinobicyclo[1.1.1]pentan-1-yl)-1H-imidazol-4-yl)-3-(trifluoromethoxy)-pyridin-2-amine hydrogen maleate.

### Embodiment 51

The compound of Embodiment 50, characterized by the presence of four or more XRPD peaks with d-spacings of about 24.26, 7.77, 5.52, 4.76, 4.52, 4.36, 4.27, 4.09, 3.97, and 3.91 Å.

### Embodiment 52

The compound of Embodiment 51, characterized by the presence of five or more XRPD peaks with d-spacings of about 24.26, 7.77, 5.52, 4.76, 4.52, 4.36, 4.27, 4.09, 3.97, and 3.91 Å.

### Embodiment 53

The compound of Embodiment 52, characterized by the presence of six or more XRPD peaks with d-spacings of about 24.26, 7.77, 5.52, 4.76, 4.52, 4.36, 4.27, 4.09, 3.97, and 3.91 Å.

### Embodiment 54

The compound of Embodiment 53, characterized by the presence of seven or more XRPD peaks with d-spacings of about 24.26, 7.77, 5.52, 4.76, 4.52, 4.36, 4.27, 4.09, 3.97, and 3.91 Å.

### Embodiment 55

The compound of Embodiment 54, characterized by the presence of eight or more XRPD peaks with d-spacings of about 24.26, 7.77, 5.52, 4.76, 4.52, 4.36, 4.27, 4.09, 3.97, and 3.91 Å.

### Embodiment 56

The compound of Embodiment 50, characterized by the presence of four or more XRPD peaks with 2-theta values, using CuKα radiation, of about 3.64, 11.38, 16.06, 18.62, 19.61, 20.36, 20.79, 21.74, 22.39, and 22.71 degrees.

### Embodiment 57

The compound of Embodiment 56, characterized by the presence of five or more XRPD peaks with 2-theta values, using CuKα radiation, of about 3.64, 11.38, 16.06, 18.62, 19.61, 20.36, 20.79, 21.74, 22.39, and 22.71 degrees.

### Embodiment 58

The compound of Embodiment 57, characterized by the presence of six or more XRPD peaks with 2-theta values, using CuKα radiation, of about 3.64, 11.38, 16.06, 18.62, 19.61, 20.36, 20.79, 21.74, 22.39, and 22.71 degrees.

### Embodiment 59

The compound of Embodiment 58, characterized by the presence of seven or more XRPD peaks with 2-theta values, using CuKα radiation, of about 3.64, 11.38, 16.06, 18.62, 19.61, 20.36, 20.79, 21.74, 22.39, and 22.71 degrees.

### Embodiment 60

The compound of Embodiment 59, characterized by the presence of eight or more XRPD peaks with 2-theta values, using CuKα radiation, of about 3.64, 11.38, 16.06, 18.62, 19.61, 20.36, 20.79, 21.74, 22.39, and 22.71 degrees.

### Embodiment 61

The compound of any one of Embodiments 50 through 60, characterized by the presence of an exothermic peak in the DSC at about 175° C.

### Embodiment 62

The compound of any one of Embodiments 50 through 60, characterized by a weight loss of no more than 2.5% below 120° C. by TGA.

### Embodiment 63

The compound of Embodiment 10, wherein the compound is 5-(2-isopropyl-1-(3-morpholinobicyclo[1.1.1]pentan-1-yl)-1H-imidazol-4-yl)-3-(trifluoromethoxy)-pyridin-2-amine hydrogen succinate.

### Embodiment 64

The compound of Embodiment 63, characterized by the presence of four or more XRPD peaks with d-spacings of about 26.09, 24.12, 10.94, 6.25, 5.49, 5.10, 4.72, 4.59, 4.26, and 4.21 Å.

### Embodiment 65

The compound of Embodiment 64, characterized by the presence of five or more XRPD peaks with d-spacings of about 26.09, 24.12, 10.94, 6.25, 5.49, 5.10, 4.72, 4.59, 4.26, and 4.21 Å.

### Embodiment 66

The compound of Embodiment 65, characterized by the presence of six or more XRPD peaks with d-spacings of about 26.09, 24.12, 10.94, 6.25, 5.49, 5.10, 4.72, 4.59, 4.26, and 4.21 Å.

### Embodiment 67

The compound of Embodiment 66, characterized by the presence of seven or more XRPD peaks with d-spacings of about 26.09, 24.12, 10.94, 6.25, 5.49, 5.10, 4.72, 4.59, 4.26, and 4.21 Å.

### Embodiment 68

The compound of Embodiment 67, characterized by the presence of eight or more XRPD peaks with d-spacings of about 26.09, 24.12, 10.94, 6.25, 5.49, 5.10, 4.72, 4.59, 4.26, and 4.21 Å.

### Embodiment 69

The compound of Embodiment 63, characterized by the presence of four or more XRPD peaks with 2-theta values, using CuKα radiation, of about 3.38, 3.66, 8.07, 14.16, 16.12, 17.37, 18.80, 19.34, 20.83, and 21.11 degrees.

### Embodiment 70

The compound of Embodiment 69, characterized by the presence of five or more XRPD peaks with 2-theta values, using CuK radiation, of about 3.38, 3.66, 8.07, 14.16, 16.12, 17.37, 18.80, 19.34, 20.83, and 21.11 degrees.

### Embodiment 71

The compound of Embodiment 70, characterized by the presence of six or more XRPD peaks with 2-theta values, using CuK radiation, of about 3.38, 3.66, 8.07, 14.16, 16.12, 17.37, 18.80, 19.34, 20.83, and 21.11 degrees.

### Embodiment 72

The compound of Embodiment 71, characterized by the presence of seven or more XRPD peaks with 2-theta values, using CuK radiation, of about 3.38, 3.66, 8.07, 14.16, 16.12, 17.37, 18.80, 19.34, 20.83, and 21.11 degrees.

### Embodiment 73

The compound of Embodiment 72, characterized by the presence of eight or more XRPD peaks with 2-theta values, using CuK radiation, of about 3.38, 3.66, 8.07, 14.16, 16.12, 17.37, 18.80, 19.34, 20.83, and 21.11 degrees.

### Embodiment 74

The compound of any one of Embodiments 63 through 73, characterized by a weight loss of no more than 0.2% below 120° C. by TGA.

### Embodiment 75

The compound of Embodiment 10, wherein the compound is 5-(2-isopropyl-1-(3-morpholinobicyclo[1.1.1]pentan-1-yl)-1H-imidazol-4-yl)-3-(trifluoromethoxy)-pyridin-2-amine hydrogen fumarate.

### Embodiment 76

The compound of Embodiment 75, characterized by the presence of four or more XRPD peaks with d-spacings of about 26.87, 24.26, 12.00, 6.33, 6.02, 5.76, 4.63, 4.16, 4.10, and 4.02 Å.

### Embodiment 77

The compound of Embodiment 76, characterized by the presence of five or more XRPD peaks with d-spacings of about 26.87, 24.26, 12.00, 6.33, 6.02, 5.76, 4.63, 4.16, 4.10, and 4.02 Å.

### Embodiment 78

The compound of Embodiment 77, characterized by the presence of six or more XRPD peaks with d-spacings of about 26.87, 24.26, 12.00, 6.33, 6.02, 5.76, 4.63, 4.16, 4.10, and 4.02 Å.

### Embodiment 79

The compound of Embodiment 78, characterized by the presence of seven or more XRPD peaks with d-spacings of about 26.87, 24.26, 12.00, 6.33, 6.02, 5.76, 4.63, 4.16, 4.10, and 4.02 Å.

### Embodiment 80

The compound of Embodiment 79, characterized by the presence of eight or more XRPD peaks with d-spacings of about 26.87, 24.26, 12.00, 6.33, 6.02, 5.76, 4.63, 4.16, 4.10, and 4.02 Å.

### Embodiment 81

The compound of Embodiment 75, characterized by the presence of four or more XRPD peaks with 2-theta values, using CuKα radiation, of about 3.29, 3.64, 7.36, 13.99, 14.70, 15.37, 19.13, 21.36, 21.68, and 22.08 degrees.

### Embodiment 82

The compound of Embodiment 81, characterized by the presence of five or more XRPD peaks with 2-theta values, using CuKα radiation, of about 3.29, 3.64, 7.36, 13.99, 14.70, 15.37, 19.13, 21.36, 21.68, and 22.08 degrees.

### Embodiment 83

The compound of Embodiment 82, characterized by the presence of six or more XRPD peaks with 2-theta values, using CuKα radiation, of about 3.29, 3.64, 7.36, 13.99, 14.70, 15.37, 19.13, 21.36, 21.68, and 22.08 degrees.

### Embodiment 84

The compound of Embodiment 83, characterized by the presence of seven or more XRPD peaks with 2-theta values, using CuKα radiation, of about 3.29, 3.64, 7.36, 13.99, 14.70, 15.37, 19.13, 21.36, 21.68, and 22.08 degrees.

### Embodiment 85

The compound of Embodiment 84, characterized by the presence of eight or more XRPD peaks with 2-theta values, using CuKα radiation, of about 3.29, 3.64, 7.36, 13.99, 14.70, 15.37, 19.13, 21.36, 21.68, and 22.08 degrees.

### Embodiment 86

The compound of any one of Embodiments 75 through 85, characterized by the presence of an exothermic peak in the DSC at about 175° C.

### Embodiment 87

The compound of any one of Embodiments 75 through 86, characterized by the presence of an endothermic peak in the DSC at about 90° C.

### Embodiment 88

The compound of any one of Embodiments 75 through 87, characterized by a weight loss of no more than 0.5% below 120° C. by TGA.

### Embodiment 89

The compound of any one of Embodiments 75 through 88, characterized by a weight loss of no more than 0.2% below 120° C. by TGA.

Also provided herein is Embodiment 90: a compound of Formula III:

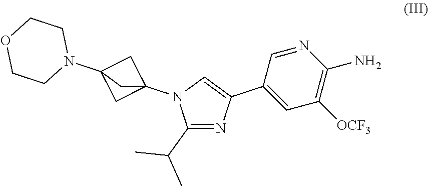 [SMILES: CC(C)c1nc(-c2cnc(N)c(OC(F)(F)F)c2)cn1C12CC(N3CCOCC3)(C1)C2]

wherein the compound is Polymorph A.

In certain embodiments of Polymorph “A”, the polymorph is characterized as having four or more peaks with a d-spacing from 24.93, 9.35, 8.25, 6.43, 5.63, 5.31, 4.86, 4.61, 4.52, 4.28, 3.97, 3.85, 3.64, 3.52, and 3.43 Å. In certain embodiments of Polymorph “A”, the polymorph is characterized as having five or more peaks with a d-spacing from 24.93, 9.35, 8.25, 6.43, 5.63, 5.31, 4.86, 4.61, 4.52, 4.28, 3.97, 3.85, 3.64, 3.52, and 3.43 Å. In certain embodiments of Polymorph “A”, the polymorph is characterized as having six or more peaks with a d-spacing from 24.93, 9.35, 8.25, 6.43, 5.63, 5.31, 4.86, 4.61, 4.52, 4.28, 3.97, 3.85, 3.64, 3.52, and 3.43 Å. In certain embodiments of Polymorph “A”, the polymorph is characterized as having seven or more peaks with a d-spacing from 24.93, 9.35, 8.25, 6.43, 5.63, 5.31, 4.86, 4.61, 4.52, 4.28, 3.97, 3.85, 3.64, 3.52, and 3.43 Å. In certain embodiments of Polymorph “A”, the polymorph is characterized as having eight or more peaks with a d-spacing from 24.93, 9.35, 8.25, 6.43, 5.63, 5.31, 4.86, 4.61, 4.52, 4.28, 3.97, 3.85, 3.64, 3.52, and 3.43 Å.

Also provided herein is Embodiment 91: a compound of Formula III:

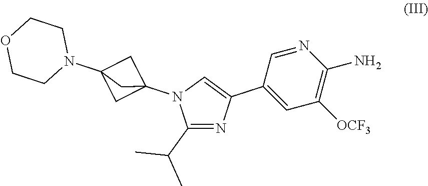 [SMILES: CC(C)c1nc(-c2cnc(N)c(OC(F)(F)F)c2)cn1C12CC(N3CCOCC3)(C1)C2]

having polymorph structure “B”.

In certain embodiments of Polymorph “B”, the polymorph is characterized as having four or more peaks with a d-spacing from 25.07, 9.43, 8.28, 7.27, 6.42, 5.64, 5.32, 4.89, 4.61, 4.52, 4.26, 3.98, 3.65, 3.52, and 2.33 Å. In certain embodiments of Polymorph “B”, the polymorph is characterized as having five or more peaks with a d-spacing from 25.07, 9.43, 8.28, 7.27, 6.42, 5.64, 5.32, 4.89, 4.61, 4.52, 4.26, 3.98, 3.65, 3.52, and 2.33 Å. In certain embodiments of Polymorph “B”, the polymorph is characterized as having six or more peaks with a d-spacing from 25.07, 9.43, 8.28, 7.27, 6.42, 5.64, 5.32, 4.89, 4.61, 4.52, 4.26, 3.98, 3.65, 3.52, and 2.33 Å. In certain embodiments of Polymorph “B”, the polymorph is characterized as having seven or more peaks with a d-spacing from 25.07, 9.43, 8.28, 7.27, 6.42, 5.64, 5.32, 4.89, 4.61, 4.52, 4.26, 3.98, 3.65, 3.52, and 2.33 Å. In certain embodiments of Polymorph “B”, the polymorph is characterized as having eight or more peaks with a d-spacing from 25.07, 9.43, 8.28, 7.27, 6.42, 5.64, 5.32, 4.89, 4.61, 4.52, 4.26, 3.98, 3.65, 3.52, and 2.33 Å.

Also provided herein is Embodiment 92: a compound of Formula III:

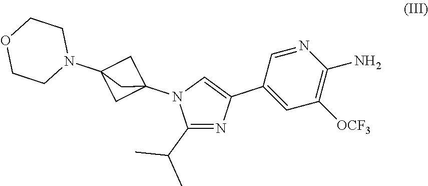 [SMILES: CC(C)c1nc(-c2cnc(N)c(OC(F)(F)F)c2)cn1C12CC(N3CCOCC3)(C1)C2]

having polymorph structure “C”.

In certain embodiments of Polymorph “C”, the polymorph is characterized as having four or more peaks with a d-spacing from 26.71, 24.93, 8.70, 6.99, 6.70, 5.35, 4.87, 4.80, 4.66, 4.60, 4.49, 4.04, 3.72, 3.60, and 3.24 Å. In certain embodiments of Polymorph “C”, the polymorph is characterized as having five or more peaks with a d-spacing from 26.71, 24.93, 8.70, 6.99, 6.70, 5.35, 4.87, 4.80, 4.66, 4.60, 4.49, 4.04, 3.72, 3.60, and 3.24 Å. In certain embodiments of Polymorph “C”, the polymorph is characterized as having six or more peaks with a d-spacing from 26.71, 24.93, 8.70, 6.99, 6.70, 5.35, 4.87, 4.80, 4.66, 4.60, 4.49, 4.04, 3.72, 3.60, and 3.24 Å. In certain embodiments of Polymorph “C”, the polymorph is characterized as having seven or more peaks with a d-spacing from 26.71, 24.93, 8.70, 6.99, 6.70, 5.35, 4.87, 4.80, 4.66, 4.60, 4.49, 4.04, 3.72, 3.60, and 3.24 Å. In certain embodiments of Polymorph “C”, the polymorph is characterized as having eight or more peaks with a d-spacing from 26.71, 24.93, 8.70, 6.99, 6.70, 5.35, 4.87, 4.80, 4.66, 4.60, 4.49, 4.04, 3.72, 3.60, and 3.24 Å.

Also provided herein is Embodiment 93: a compound of Formula III:

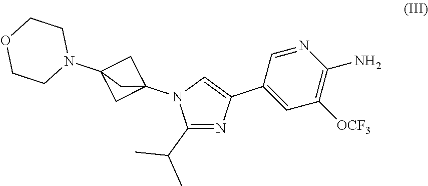 [SMILES: CC(C)c1nc(-c2cnc(N)c(OC(F)(F)F)c2)cn1C12CC(N3CCOCC3)(C1)C2]

having polymorph structure “D”.

In certain embodiments of Polymorph “D”, the polymorph is characterized as having four or more peaks with a d-spacing from 26.71, 24.93, 8.70, 6.99, 6.70, 5.35, 4.87, 4.80, 4.66, 4.60, 4.49, 4.04, 3.72, 3.60, and 3.24 Å. In certain embodiments of Polymorph “D”, the polymorph is characterized as having five or more peaks with a d-spacing from 26.71, 24.93, 8.70, 6.99, 6.70, 5.35, 4.87, 4.80, 4.66, 4.60, 4.49, 4.04, 3.72, 3.60, and 3.24 Å. In certain embodiments of Polymorph “D”, the polymorph is characterized as having six or more peaks with a d-spacing from 26.71, 24.93, 8.70, 6.99, 6.70, 5.35, 4.87, 4.80, 4.66, 4.60, 4.49, 4.04, 3.72, 3.60, and 3.24 Å. In certain embodiments of Polymorph “D”, the polymorph is characterized as having seven or more peaks with a d-spacing from 26.71, 24.93, 8.70, 6.99, 6.70, 5.35, 4.87, 4.80, 4.66, 4.60, 4.49, 4.04, 3.72, 3.60, and 3.24 Å. In certain embodiments of Polymorph “D”, the polymorph is characterized as having eight or more peaks with a d-spacing from 26.71, 24.93, 8.70, 6.99, 6.70, 5.35, 4.87, 4.80, 4.66, 4.60, 4.49, 4.04, 3.72, 3.60, and 3.24 Å.

Also provided herein is Embodiment 94: a compound of Formula III:

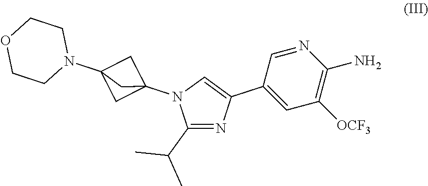 [SMILES: CC(C)c1nc(-c2cnc(N)c(OC(F)(F)F)c2)cn1C12CC(N3CCOCC3)(C1)C2]

having polymorph structure “E”.

In certain embodiments of Polymorph “E”, the polymorph is characterized as having four or more peaks with a d-spacing from 25.07, 6.98, 6.53, 6.31, 5.57, 5.36, 5.09, 4.65, 4.48, 4.27, 4.16, 4.01, 3.31, 3.28, and 2.33 Å. In certain embodiments of Polymorph “E”, the polymorph is characterized as having five or more peaks with a d-spacing from 25.07, 6.98, 6.53, 6.31, 5.57, 5.36, 5.09, 4.65, 4.48, 4.27, 4.16, 4.01, 3.31, 3.28, and 2.33 Å. In certain embodiments of Polymorph “E”, the polymorph is characterized as having six or more peaks with a d-spacing from 25.07, 6.98, 6.53, 6.31, 5.57, 5.36, 5.09, 4.65, 4.48, 4.27, 4.16, 4.01, 3.31, 3.28, and 2.33 Å. In certain embodiments of Polymorph “E”, the polymorph is characterized as having seven or more peaks with a d-spacing from 25.07, 6.98, 6.53, 6.31, 5.57, 5.36, 5.09, 4.65, 4.48, 4.27, 4.16, 4.01, 3.31, 3.28, and 2.33 Å. In certain embodiments of Polymorph “E”, the polymorph is characterized as having eight or more peaks with a d-spacing from 25.07, 6.98, 6.53, 6.31, 5.57, 5.36, 5.09, 4.65, 4.48, 4.27, 4.16, 4.01, 3.31, 3.28, and 2.33 Å.

Also provided herein is Embodiment 95: a compound of Formula III:

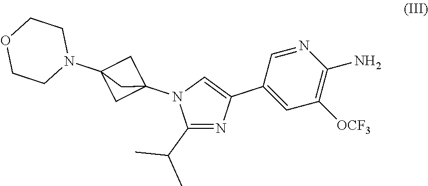 [SMILES: CC(C)c1nc(-c2cnc(N)c(OC(F)(F)F)c2)cn1C12CC(N3CCOCC3)(C1)C2]

having polymorph structure “F”.

In certain embodiments of Polymorph “F”, the polymorph is characterized as having four or more peaks with a d-spacing from 24.79, 9.33, 8.24, 7.91, 7.02, 6.89, 6.41, 4.83, 4.67, 4.56, 4.51, 4.28, 3.95, 3.87, and 3.35 Å. In certain embodiments of Polymorph “F”, the polymorph is characterized as having five or more peaks with a d-spacing from 24.79, 9.33, 8.24, 7.91, 7.02, 6.89, 6.41, 4.83, 4.67, 4.56, 4.51, 4.28, 3.95, 3.87, and 3.35 Å. In certain embodiments of Polymorph “F”, the polymorph is characterized as having six or more peaks with a d-spacing from 24.79, 9.33, 8.24, 7.91, 7.02, 6.89, 6.41, 4.83, 4.67, 4.56, 4.51, 4.28, 3.95, 3.87, and 3.35 Å. In certain embodiments of Polymorph “F”, the polymorph is characterized as having seven or more peaks with a d-spacing from 24.79, 9.33, 8.24, 7.91, 7.02, 6.89, 6.41, 4.83, 4.67, 4.56, 4.51, 4.28, 3.95, 3.87, and 3.35 Å. In certain embodiments of Polymorph “F”, the polymorph is characterized as having eight or more peaks with a d-spacing from 24.79, 9.33, 8.24, 7.91, 7.02, 6.89, 6.41, 4.83, 4.67, 4.56, 4.51, 4.28, 3.95, 3.87, and 3.35 Å.

Also provided herein is Embodiment 96: a compound of Formula III:

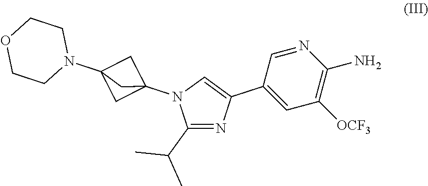 [SMILES: CC(C)c1nc(-c2cnc(N)c(OC(F)(F)F)c2)cn1C12CC(N3CCOCC3)(C1)C2]

having polymorph structure “G”.

In certain embodiments of Polymorph “G”, the polymorph is characterized as having four or more peaks with a d-spacing from 25.07, 16.44, 8.24, 6.43, 5.50, 5.32, 4.86, 4.75, 4.61, 4.54, 4.29, 4.11, 3.97, 3.41, and 3.37 Å. In certain embodiments of Polymorph “G”, the polymorph is characterized as having five or more peaks with a d-spacing from 25.07, 16.44, 8.24, 6.43, 5.50, 5.32, 4.86, 4.75, 4.61, 4.54, 4.29, 4.11, 3.97, 3.41, and 3.37 Å. In certain embodiments of Polymorph “G”, the polymorph is characterized as having six or more peaks with a d-spacing from 25.07, 16.44, 8.24, 6.43, 5.50, 5.32, 4.86, 4.75, 4.61, 4.54, 4.29, 4.11, 3.97, 3.41, and 3.37 Å. In certain embodiments of Polymorph “G”, the polymorph is characterized as having seven or more peaks with a d-spacing from 25.07, 16.44, 8.24, 6.43, 5.50, 5.32, 4.86, 4.75, 4.61, 4.54, 4.29, 4.11, 3.97, 3.41, and 3.37 Å. In certain embodiments of Polymorph “G”, the polymorph is characterized as having eight or more peaks with a d-spacing from 25.07, 16.44, 8.24, 6.43, 5.50, 5.32, 4.86, 4.75, 4.61, 4.54, 4.29, 4.11, 3.97, 3.41, and 3.37 Å.

Also provided herein is Embodiment 97: a compound of Formula III:

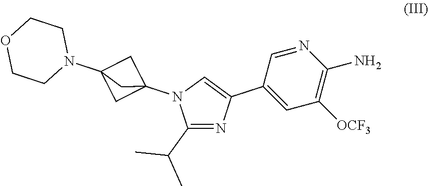 [SMILES: CC(C)c1nc(-c2cnc(N)c(OC(F)(F)F)c2)cn1C12CC(N3CCOCC3)(C1)C2]

having polymorph structure “H”.

In certain embodiments of Polymorph “H”, the polymorph is characterized as having four or more peaks with a d-spacing from 4.52, 4.68, 6.41, 4.61, 27.53, 4.28, 25.07, 9.33, 3.21, 2.31, 3.42, 3.35, 4.01, 2.73, and 3.39 Å. In certain embodiments of Polymorph “H”, the polymorph is characterized as having five or more peaks with a d-spacing from 4.52, 4.68, 6.41, 4.61, 27.53, 4.28, 25.07, 9.33, 3.21, 2.31, 3.42, 3.35, 4.01, 2.73, and 3.39 Å. In certain embodiments of Polymorph “H”, the polymorph is characterized as having six or more peaks with a d-spacing from 4.52, 4.68, 6.41, 4.61, 27.53, 4.28, 25.07, 9.33, 3.21, 2.31, 3.42, 3.35, 4.01, 2.73, and 3.39 Å. In certain embodiments of Polymorph “H”, the polymorph is characterized as having seven or more peaks with a d-spacing from 4.52, 4.68, 6.41, 4.61, 27.53, 4.28, 25.07, 9.33, 3.21, 2.31, 3.42, 3.35, 4.01, 2.73, and 3.39 Å. In certain embodiments of Polymorph “H”, the polymorph is characterized as having eight or more peaks with a d-spacing from 4.52, 4.68, 6.41, 4.61, 27.53, 4.28, 25.07, 9.33, 3.21, 2.31, 3.42, 3.35, 4.01, 2.73, and 3.39 Å.

Also provided herein is Embodiment 98: a compound of Formula III:

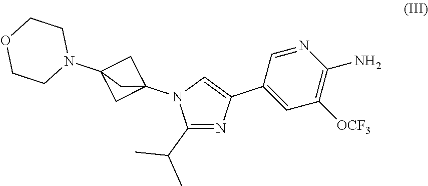 [SMILES: CC(C)c1nc(-c2cnc(N)c(OC(F)(F)F)c2)cn1C12CC(N3CCOCC3)(C1)C2]

having polymorph structure “I”.

In certain embodiments of Polymorph “I”, the polymorph is characterized as having four or more peaks with a d-spacing from 8.22, 5.50, 16.28, 4.13, 26.71, 24.80, 6.45, 3.31, 4.60, 2.76, 3.63, 5.00, 4.53, 6.97, and 6.64 Å. In certain embodiments of Polymorph “I”, the polymorph is characterized as having five or more peaks with a d-spacing from 8.22, 5.50, 16.28, 4.13, 26.71, 24.80, 6.45, 3.31, 4.60, 2.76, 3.63, 5.00, 4.53, 6.97, and 6.64 Å. In certain embodiments of Polymorph “I”, the polymorph is characterized as having six or more peaks with a d-spacing from 8.22, 5.50, 16.28, 4.13, 26.71, 24.80, 6.45, 3.31, 4.60, 2.76, 3.63, 5.00, 4.53, 6.97, and 6.64 Å. In certain embodiments of Polymorph “I”, the polymorph is characterized as having seven or more peaks with a d-spacing from 8.22, 5.50, 16.28, 4.13, 26.71, 24.80, 6.45, 3.31, 4.60, 2.76, 3.63, 5.00, 4.53, 6.97, and 6.64 Å. In certain embodiments of Polymorph “I”, the polymorph is characterized as having eight or more peaks with a d-spacing from 8.22, 5.50, 16.28, 4.13, 26.71, 24.80, 6.45, 3.31, 4.60, 2.76, 3.63, 5.00, 4.53, 6.97, and 6.64 Å.

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

Also provided is a compound chosen from the Examples disclosed herein.

The present disclosure also relates to a method of inhibiting at least one DLK function comprising the step of contacting DLK with a compound as described herein. The cell phenotype, cell proliferation, activity of DLK, change in biochemical output produced by active DLK, expression of DLK, or binding of DLK with a natural binding partner may be monitored. Such methods may be modes of treatment of disease, biological assays, cellular assays, biochemical assays, or the like.

Also provided herein is a method of treatment of a DLK-mediated disease comprising the administration of a therapeutically effective amount of a compound as disclosed herein, or a salt thereof, to a patient in need thereof.

In certain embodiments, the disease is a neurodegenerative disease.

Also provided herein is a compound as disclosed herein for use as a medicament.

Also provided herein is a compound as disclosed herein for use as a medicament for the treatment of a DLK-mediated disease.

Also provided is the use of a compound as disclosed herein as a medicament.

Also provided is the use of a compound as disclosed herein as a medicament for the treatment of a DLK-mediated disease.

Also provided is a compound as disclosed herein for use in the manufacture of a medicament for the treatment of a DLK-mediated disease.

Also provided is the use of a compound as disclosed herein for the treatment of a DLK-mediated disease.

Also provided herein is a method of inhibition of DLK comprising contacting DLK with a compound as disclosed herein, or a salt thereof.

Also provided herein is a method for achieving an effect in a patient comprising the administration of a therapeutically effective amount of a compound as disclosed herein, or a salt thereof, to a patient, wherein the effect is chosen from cognition enhancement.

In certain embodiments, the DLK-mediated disease is chosen from a disease that results from traumatic injury to central nervous system and peripheral nervous system neurons (e.g. stroke, traumatic brain injury, spinal cord injury), a disease that results from a chronic neurodegenerative condition (e.g. Alzheimer's disease, frontotemporal dementia, Parkinson's disease, Huntington's disease, amyotrophic lateral sclerosis, spinocerebellar ataxia, progressive supranuclear palsy, Lewy body disease, Kennedy's disease, and other related conditions), a disease that results from neuropathies resulting from neurological damage (chemotherapy-induced peripheral neuropathy, diabetic neuropathy, and related conditions) and a disease that results from cognitive disorders caused by pharmacological intervention (e.g. chemotherapy induced cognitive disorder, also known as chemobrain).

Also provided is a method of modulation of a DLK-mediated function in a subject comprising the administration of a therapeutically effective amount of a compound as disclosed herein.

Also provided is a pharmaceutical composition comprising a compound as disclosed herein, together with a pharmaceutically acceptable carrier.

In certain embodiments, the pharmaceutical composition is formulated for oral administration.

In certain embodiments, the pharmaceutical composition is formulated for parenteral administration.

In certain embodiments, the oral pharmaceutical composition is chosen from a tablet and a capsule.

### Abbreviations and Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, nonyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “imido”, as used herein, alone or in combination, refers to —NRR′, wherein R and R′ are both acyl, either of which may themselves be optionally substituted, and R and R′ may combine to form a saturated or unsaturated ring. Examples of compounds comprising the imido group include diacetamide, succinimide, maleimide, and phthalimide.

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group—with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or combinations thereof, fully saturated or containing from 1 to 3 degrees of unsaturation, consisting of the stated number of carbon atoms and from one to three heteroatoms chosen from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms (i.e., C₁-C₆ alkyl).

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms chosen from N, O, and S, or 2) bicyclic heteroaryl, wherein each of the fused rings comprises five or six ring members, comprising between them one to four heteroatoms chosen from N, O, and S.

The term “lower cycloalkyl,” as used herein, alone or in combination, means a monocyclic cycloalkyl having between three and six ring members (i.e., C₃-C₆ cycloalkyl). Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms chosen from N, O, and S (i.e., C₃-C₆ heterocycloalkyl). Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently chosen from hydrogen and lower alkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿwhere n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“DLK binder” is used herein to refer to a compound that exhibits a K_{d} with respect to DLK of no more than about 100 μM and more typically not more than about 50 μM, as measured in the DLK binding assay described generally herein. The DLK binding assay measures the K_{d} (dissociation constant) for the binding of a compound with the active site of DLK. Certain compounds disclosed herein have been discovered to bind to DLK. In certain embodiments, compounds will exhibit an K_{d} with respect to DLK of no more than about 10 μM; in further embodiments, compounds will exhibit a K_{d} with respect to DLK of no more than about 1 μM; in yet further embodiments, compounds will exhibit a K_{d} with respect to DLK of not more than about 0.1 μM; in yet further embodiments, compounds will exhibit a K_{d} with respect to DLK of not more than about 10 nM, as measured in the DLK assay described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, hydrogen citrate, dihydrogen citrate, digluconate, formate, fumarate, hydrogen fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, hydrogen maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, hydrogen succinate, sulfonate, tartrate, hydrogen tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

Without intending to be limiting, salts comprising the following anions are contemplated in this disclosure:

TABLE 1
Anions of the disclosure

| Name | Empirical Formula |
|---|---|
| sulfate | SO₄²⁻ |
| hydrogen sulfate | HSO₄⁻ |
| phosphate | PO₄³⁻ |
| hydrogen phosphate | HPO₄²⁻ |
| dihydrogen phosphate | H₂PO₄⁻ |
| citrate | C₆H₅O₇³⁻ |
| hydrogen citrate | C₆H₆O₇²⁻ |
| dihydrogen citrate | C₆H₇O₇⁻ |
| succinate | C₄H₄O₄²⁻ |
| hydrogen succinate | C₄H₅O₄⁻ |
| maleate | C₄H₂O₄²⁻ |
| hydrogen maleate | C₄H₃O₄⁻ |
| fumarate | C₄H₂O₄²⁻ |
| hydrogen fumarate | C₄H₃O₄⁻ |
| tartrate | C₄H₄O₆²⁻ |
| hydrogen tartrate | C₄H₅O₆⁻ |
| malate | C₄H₂O₅²⁻ |
| hydrogen malate | C₄H₃O₅⁻ |

### Pharmaceutical Compositions

While it may be possible for the compounds of the subject disclosure to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject disclosure or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

### Administration and Treatment

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

### Oral Administration

The compounds of the present disclosure may be administered orally, including swallowing, so the compound enters the gastrointestinal tract, or is absorbed into the blood stream directly from the mouth, including sublingual or buccal administration.

Suitable compositions for oral administration include solid formulations such as tablets, pills, cachets, lozenges and hard or soft capsules, which can contain liquids, gels, powders, or granules, solutions or suspensions in an aqueous liquid or a non-aqueous liquid, or as an oil-in-water liquid emulsion or a water-in-oil liquid emulsion. The active ingredient may also be presented as a bolus, electuary or paste.

In a tablet or capsule dosage form the amount of drug present may be from about 0.05% to about 95% by weight, more typically from about 2% to about 50% by weight of the dosage form.

In addition, tablets or capsules may contain a disintegrant, comprising from about 0.5% to about 35% by weight, more typically from about 2% to about 25% of the dosage form. Examples of disintegrants include methyl cellulose, sodium or calcium carboxymethyl cellulose, croscarmellose sodium, polyvinylpyrrolidone, hydroxypropyl cellulose, starch and the like.

Suitable binders, for use in a tablet, include gelatin, polyethylene glycol, sugars, gums, starch, hydroxypropyl cellulose and the like. Suitable diluents, for use in a tablet, include mannitol, xylitol, lactose, dextrose, sucrose, sorbitol and starch.

Suitable surface active agents and glidants, for use in a tablet or capsule, may be present in amounts from about 0.1% to about 3% by weight, and include polysorbate 80, sodium dodecyl sulfate, talc and silicon dioxide.

Suitable lubricants, for use in a tablet or capsule, may be present in amounts from about 0.1% to about 5% by weight, and include calcium, zinc or magnesium stearate, sodium stearyl fumarate and the like.

Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with a liquid diluent. Dyes or pigments may be added to tablets for identification or to characterize different combinations of active compound doses.

Liquid formulations can include emulsions, solutions, syrups, elixirs and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

Compositions for oral administration may be formulated as immediate or modified release, including delayed or sustained release, optionally with enteric coating.

In another embodiment, a pharmaceutical composition comprises a therapeutically effective amount of a compound of Formula (I) or a pharmaceutically acceptable salt thereof, and a pharmaceutically acceptable carrier.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

### Parenteral Administration

Compounds of the present disclosure may be administered directly into the blood stream, muscle, or internal organs by injection, e.g., by bolus injection or continuous infusion. Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods. The formulations may be presented in unit-dose or multi-dose containers, for example sealed ampoules and vials.

Most parenteral formulations are aqueous solutions containing excipients, including salts, buffering, suspending, stabilizing and/or dispersing agents, antioxidants, bacteriostats, preservatives, and solutes which render the formulation isotonic with the blood of the intended recipient, and carbohydrates.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions. Compositions for parenteral administration may be formulated as immediate or modified release, including delayed or sustained release. Compounds may also be formulated as depot preparations. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

### Topical Administration

Compounds of the present disclosure may be administered topically (for example to the skin, mucous membranes, ear, nose, or eye) or transdermally. Formulations for topical administration can include, but are not limited to, lotions, solutions, creams, gels, hydrogels, ointments, foams, implants, patches and the like. Carriers that are pharmaceutically acceptable for topical administration formulations can include water, alcohol, mineral oil, glycerin, polyethylene glycol and the like. Topical administration can also be performed by, for example, electroporation, iontophoresis, phonophoresis and the like.

Typically, the active ingredient for topical administration may comprise from 0.001% to 10% w/w (by weight) of the formulation. In certain embodiments, the active ingredient may comprise as much as 10% w/w; less than 5% w/w; from 2% w/w to 5% w/w; or from 0.1% to 1% w/w of the formulation.

Compositions for topical administration may be formulated as immediate or modified release, including delayed or sustained release.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

### Rectal, Buccal, and Sublingual Administration

Suppositories for rectal administration of the compounds of the present disclosure can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

### Administration by Inhalation

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Other carrier materials and modes of administration known in the pharmaceutical art may also be used. Pharmaceutical compositions of the disclosure may be prepared by any of the well-known techniques of pharmacy, such as effective formulation and administration procedures. Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

### Combinations and Combination Therapy

In certain instances, it may be appropriate to administer at least one of the compounds described herein (or a pharmaceutically acceptable salt thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds of the disclosure with: donepezil, rivastigmine, galantamine, and memantine. Further examples include anti-amyloid antibodies and vaccines, anti-Ab antibodies and vaccines, anti-tau antibodies and vaccines, β-secretase inhibitors, 5-HT4 agonists, 5-HT6 antagonists, 5-HT1a antagonists, α7 nicotinic receptor agonists, 5-HT3 receptor antagonists, PDE4 inhibitors, O-GlcNAcase inhibitors, and other medicines approved for the treatment of Alzheimer's disease. Further examples include metformin, minocycline, tissue plasminogen activator, and other therapies that improve neuronal survival.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating DLK-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein, in combination with one or more additional agents for the treatment of DLK-mediated disorders.

In certain embodiments, the compounds, compositions, and methods disclosed herein may be useful for the treatment of neurological diseases that result from traumatic injury to central nervous system and peripheral nervous system neurons.

In certain embodiments, the compounds, compositions, and methods disclosed herein may be useful for the treatment of stroke.

In certain embodiments, the compounds, compositions, and methods disclosed herein may be useful for the treatment of traumatic brain injury.

In certain embodiments, the compounds, compositions, and methods disclosed herein may be useful for the treatment of spinal cord injury.

In certain embodiments, the compounds, compositions, and methods disclosed herein may be useful for the treatment of neurologic diseases that result from a chronic neurodegenerative condition.

In certain embodiments, the neurodegenerative condition is Alzheimer's disease.

In certain embodiments, the neurodegenerative condition is frontotemporal dementia.

In certain embodiments, the neurodegenerative condition is Parkinson's disease.

In certain embodiments, the neurodegenerative condition is Huntington's disease.

In certain embodiments, the neurodegenerative condition is amyotrophic lateral sclerosis.

In certain embodiments, the neurodegenerative condition is Alzheimer's disease.

In certain embodiments, the neurodegenerative condition is spinocerebellar ataxia.

In certain embodiments, the neurodegenerative condition is progressive supranuclear palsy.

In certain embodiments, the neurodegenerative condition is Lewy body disease.

In certain embodiments, the neurodegenerative condition is Kennedy's disease.

In certain embodiments, the compounds, compositions, and methods disclosed herein may be useful for the treatment of neuropathies resulting from neural damage.

In certain embodiments, the neuropathy is chemotherapy-induced peripheral neuropathy.

In certain embodiments, the neuropathy is diabetic neuropathy.

In certain embodiments, the compounds, compositions, and methods disclosed herein may be useful for the treatment of cognitive disorders.

In certain embodiments, the cognitive disorder is caused by pharmacological intervention.

In certain embodiments, the cognitive disorder is chemotherapy induced cognitive disorder.

In certain embodiments, the compounds, compositions, and methods disclosed herein may be coadministered with another therapeutic agent.

In certain embodiments, the compounds, compositions, and methods disclosed herein may be coadministered with another therapeutic agent for the treatment of cognitive disorders.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

Compound Synthesis

Compounds of the present disclosure can be prepared using methods illustrated in general synthetic schemes and experimental procedures detailed below. General synthetic schemes and experimental procedures are presented for purposes of illustration and are not intended to be limiting. Starting materials used to prepare compounds of the present disclosure are commercially available or can be prepared using routine methods known in the art.

### List of Abbreviations

Ac₂O=acetic anhydride; AcCl=acetyl chloride; AcOH=acetic acid; AIBN=azobisisobutyronitrile; Alloc=(allyloxy)carbonyl aq.=aqueous; Boc=(tert-butyloxy)carbonyl; Bu₃SnH=tributyltin hydride; CBz=(benzyloxy)carbonyl; CD₃OD=deuterated methanol; CDCl₃=deuterated chloroform; CDI=1,1′-Carbonyldiimidazole; DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; DPPA=diphenylphosphoryl azide; EDC.HCl=EDCI.HCl=1-ethyl-3-(3-dimethylaminopropyl)carbodiimide hydrochloride; Et₂O=diethyl ether; EtOAc=ethyl acetate; EtOH=ethanol; h=hour; FaSSIF=fasted state simulated intestinal fluid; FeSSIF=fed state simulated intestinal fluid; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; i-PrOH=isopropanol; LAH=lithium aluminium hydride; LiHMDS=Lithium bis(trimethylsilyl)amide; MeCN=acetonitrile; MeOH=methanol; MP-carbonate resin=macroporous triethylammonium methylpolystyrene carbonate resin; MsCl=mesyl chloride; MTBE=methyl tertiary butyl ether; MW=microwave irradiation; n-BuLi=n-butyllithium; NaHMDS=Sodium bis(trimethylsilyl)amide; NaOMe=sodium methoxide; NaOtBu=sodium t-butoxide; NBS=N-bromosuccinimide; NCS=N-chloro-succinimide; NMP=N-Methyl-2-pyrrolidone; Pd(Ph₃)₄=tetrakis(triphenylphosphine)-palladium(0); Pd₂(dba)₃=tris(dibenzylideneacetone)dipalladium(0); PdCl₂(PPh₃)₂=bis(triphenylphosphine)palladium(II) dichloride; PG=protecting group; prep-HPLC=preparative high-performance liquid chromatography; PyBop=(benzotriazol-1-yloxy)-tripyrrolidinophosphonium hexafluorophosphate; Pyr=pyridine; RT=room temperature; RuPhos=2-dicyclohexylphosphino-2′,6′-diisopropoxybiphenyl; sat.=saturated; ss=saturated solution; SGF=simulated gastric fluid; t-BuOH=tert-butanol; T3P=Propylphosphonic Anhydride; TBS=TBDMS=tert-butyldimethylsilyl; TBSCl=TBDMSCl=tert-butyldimethylchlorosilane; TEA=Et₃N=triethylamine; Teoc=(2-trimethylsilylethoxy)-carbonyl; TFA=trifluoroacetic acid; TFAA=trifluoroacetic anhydride; THF=tetrahydrofuran; Tol=toluene; Troc=(2,2,2-trichloroethoxy)carbonyl TsC1=tosyl chloride; XPhos=2-dicyclohexylphosphino-2′,4′,6′-triisopropylbiphenyl.

### Example 1: 5-(2-isopropyl-1-(3-morpholinobicyclo[1.1.1]pentan-1-yl)-1H-imidazol-4-yl)-3-(trifluoromethoxy)pyridin-2-amine

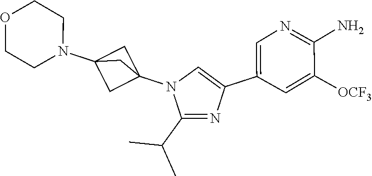 [SMILES: CC(C)c1nc(-c2cnc(N)c(OC(F)(F)F)c2)cn1C12CC(N3CCOCC3)(C1)C2]

### Step 1: tert-Butyl (3-(2-isopropyl-1H-imidazol-1-yl)bicyclo[1.1.1]pentan-1-yl)carbamate

To a solution of isobutyraldehyde (14.55 g, 201.8 mmol) in MeOH (500 ml) were added tert-butyl (3-aminobicyclo[1.1.1]pentan-1-yl)carbamate (40 g, 200 mmol), NH₄OAc (15.55 g, 201.7 mmol) and 40% aq. glyoxal (29.3 g, 202 mmol). The mixture was stirred at 25° C. for 16 h, then concentrated under reduced pressure. To the residue was added sat. aq. NaHCO₃ (500 mL) and the mixture was extracted with EtOAc (3×300 mL). The combined organic layers were washed with sat. aq. NaCl, dried over MgSO₄, filtered and concentrated under reduced pressure to give the crude title compound (52.5 g, 89%), which was used without further purification. MS (ES⁺) C₁₆H₂₅N₃O₂ requires: 291, found: 292 [M+H]⁺.

### Step 2: tert-Butyl (3-(4,5-diiodo-2-isopropyl-1H-imidazol-1-yl)bicyclo[1.1.1]pentan-1-yl)carbamate

To a solution of the product from the previous step (52.5 g, 180 mmol) in DMF (200 ml) was added NIS (122 g, 541 mmol) and the resulting mixture was stirred at 80° C. for 1 h. To the mixture were added water (1000 ml) and sat. aq. Na₂S₂O₃ (100 ml), and the mixture was extracted with EtOAc (3×300 mL). The combined organic layers were washed with sat. aq. NaCl, dried over MgSO₄, filtered and concentrated under reduced pressure. The residue was purified by SiO₂ gel chromatography (0 to 40% EtOAc in hexanes) to give the title compound as a white foamy solid (29.8 g, 30%). MS (ES⁺) C₁₆H₂₃I₂N₃O₂ requires: 543, found: 544 [M+H]⁺.

### Step 3: tert-butyl (3-(4-iodo-2-isopropyl-1H-imidazol-1-yl)bicyclo[1.1.1]pentan-1-yl)carbamate

To a solution of the product from the previous step (29.8 g, 54.9 mmol) in THF (300 ml) was added a 2.0 M iPrMgCl in THF solution (35.7 ml, 71.3 mmol), and the resulting mixture was stirred at −78° C. for 1 h then treated with sat. aq. NH₄Cl (500 mL). The aqueous phase was extracted with EtOAc (3×200 mL), and the combined organic layers were washed with sat. aq. NaCl, dried over MgSO₄, filtered and concentrated under reduced pressure. The residue was purified by SiO₂ gel chromatography (0 to 50% EtOAc in hexanes) to give the title compound as a white solid (21.4 g, 93%). MS (ES⁺) C₁₆H₂₄IN₃O₂ requires: 417, found: 418 [M+H]⁺.

### Step 4: 3-(4-iodo-2-isopropyl-1H-imidazol-1-yl)bicyclo[1.1.1]pentan-1-amine dihydrochloride

AcCl (72.9 ml, 1030 mmol) was added dropwise to MeOH (300 ml). The resulting solution was allowed to cool to RT, then added to a flask containing the product from the previous step (21.4 g, 51.3 mmol) The resulting mixture was stirred at RT for 6 h, then concentrated under reduced pressure to give the crude title compound as a white solid (22.3 g, 111%), which was used without further purification. MS (ES⁺) C₁₁H₁₆IN₃ requires: 317, found: 318 [M+H]⁺.

### Step 5: 4-(3-(4-iodo-2-isopropyl-1H-imidazol-1-yl)bicyclo[1.1.1]pentan-1-yl)morpholine

To a solution of the product from the previous step (20 g, 51 mmol) in MeCN (300 ml) were added 1-bromo-2-(2-bromoethoxy)ethane (35.7 g, 154 mmol) and K₂CO₃ (28.4 g, 205 mmol), and the resulting mixture was stirred at 90° C. for 16 h. The mixture was allowed to cool to RT, then filtered through CELITE® and the filtrate was concentrated under reduced pressure. The residue was purified by SiO₂ gel chromatography (0 to 4% MeOH in DCM) to give the title compound as a white solid (16.1 g, 81%). MS (ES⁺) C₁₅H₂₂IN₃O requires: 387, found: 388 [M+H]⁺.

### Step 6: 5-(2-isopropyl-1-(3-morpholinobicyclo[1.1.1]pentan-1-yl)-1H-imidazol-4-yl)-3-(trifluoromethoxy)pyridin-2-amine

A degassed solution of the product from the previous step (16.1 g, 41.6 mmol), 5-(4,4,5,5-tetramethyl-1,3,2-dioxaborolan-2-yl)-3-(trifluoromethoxy)pyridin-2-amine (12.6 g, 41.6 mmol), PdCl₂(dppf)-CH₂Cl₂ (1.698 g, 2.079 mmol) and 2.0 M aq. K₂CO₃ (41.6 ml, 83 mmol) in DMF (100 ml) was stirred at 90° C. for 2 h. The mixture was allowed to cool to RT, then water (1000 ml) and 1 M aq. HCl (100 mL) were added. The aqueous phase was extracted with EtOAc (3×300 mL). The aqueous layer was adjusted to pH 8-9 using 10% aq. NaOH, then extracted with EtOAc (3×300 ml). The second set of combined organic layers were washed with sat. aq. NaCl, dried over MgSO₄, filtered and concentrated under reduced pressure. The residue was purified by SiO₂ gel chromatography (0 to 10% MeOH in DCM) to give the title compound as a white solid (14.2 g, 78%). MS (ES⁺) C₂₁H₂₆F₃N₅O₂ requires: 437, found: 438 [M+H]⁺. ¹H NMR (CD₃OD) δ 8.28 (d, 1H, J=2.0 Hz), 7.90 (s, 1H), 7.77 (s, 1H), 3.78 (t, 4H, J=4.7 Hz), 3.64-3.58 (m, 1H), 2.71 (t, 4H, J=4.7 Hz), 2.60 (s, 6H), 1.46 (d, 6H, J=7.0 Hz).

### Methods

Polarized light microscopy was performed with Nikon LV100POL with a 5 megapixel CCD camera and physical lens of 20× magnification.

X-ray powder diffraction (“XRPD”) was performed with a Bruker D8 ADVANCE using CuKα radiation operating at 40 kV/40 mA, scan range 3° to 40°, sample rotation=15 rpm, scan rate=10°/min.

Differential scanning calorimetry (“DSC”) was performed with a TA Q2000 instrument, using approx 1 mg of sample in a crimped Al pan, heating from room temperature to 300° C. at a rate of 10° C./min.

Thermogravimetric analysis (“TGA”) was performed with a TA Q5000 instrument, using approx 2-5 mg of sample in an open Pt pan, heating from room temperature to 300° C. at a rate of 10° C./min.

Dynamic vapor sorption (“DVS”) was performed at 25° C., using approx 10-15 mg of sample and N₂ gas at a flow rate of 200 mL/min, dm/dt=0.01% min, minimum dm/dt stability duration of 10 min, max equilibration time of 180 min, drying at 0% RH for 120 min, scanning from 0% RH to 90% RH to 0% RH with a step size of 10% RH.

HPLC was performed with a Waters Symmetry C18 column at 40° C., flow rate of 1 mL/min, detection at 265 nm, injection volume 5 uL diluted in 1:1 CH₃CN:H₂O. Mobile phase “A”=0.1% TFA/H₂O; Mobile phase “B”=CH₃CN, gradient as follows:

TABLE 2
HPLC gradient program

| Time (min) | Mobile phase A, % | Mobile phase B, % |
|---|---|---|
| 0.00 | 80 | 20 |
| 0.01 | 80 | 20 |
| 10.00 | 10 | 90 |
| 10.10 | 80 | 20 |

### Characterization of the Example 1 Compound

The Example 1 compound is a white powder which is birefringent from PLM, and with weak crystalline peaks in the XRPD. DSC showed a single endothermic peak with an onset of 238.83° C. (71.67 J/g), and a single exothermic peak with an onset of 240.99° C. (628.3 J/g). TGA showed a weight loss of 0.2875% from 35.61° C. to 120.21° C.

The Example 1 compound was analyzed by HPLC. The purity of the material was estimated at 99.56%.

Solubility of the Example 1 compound in various organic solvents was determined by the following procedure. About 25 mg of the Example 1 compound was combined with 0.5 mL of a selected organic solvent in a 2.0 mL vial. The mixture was stirred at 800 rpm at 25° C. for 2 hr. If the compound was not dissolved completely by this time, the temperature was then raised to 50° C. and stirring at 800 rpm was continued overnight. If a solid precipitate was present, the solid precipitate was then removed by centrifugation (14,000 RPM, 5 min) and dried under vacuum at 30° C. (Method I). If a solid precipitate was not present, the solvent was removed by evaporation under vacuum at 30° C. (Method II). In either case, the resulting material was evaluated by XRPD.

TABLE 3
Solubility of the Example 1 compound in organic solvents

| | Solubility | | | Solubility | |
|---|---|---|---|---|---|
| Solvent | RT | 50° C. | Solvent | RT | 50° C. |
| MeOH | >50 | N/A | DMF | >50 | N/A |
| EtOH | <50 | >50 | DMSO | >50 | N/A |
| IPA | >50 | N/A | DCM | >50 | N/A |
| ACN | <50 | >50 | Toluene | <50 | <50 |
| Acetone | >50 | N/A | Heptane | <50 | <50 |
| MEK | >50 | N/A | H₂O | <50 | <50 |
| MTBE | <50 | <50 | Dioxane | >50 | N/A |
| EtOAc | >50 | N/A | 1/1 (v/v) EtOH/ | <50 | <50 |
| | | | H₂O | | |
| THF | >50 | N/A | 1/1 (v/v) Acetone/ | <50 | <50 |
| | | | H₂O | | |

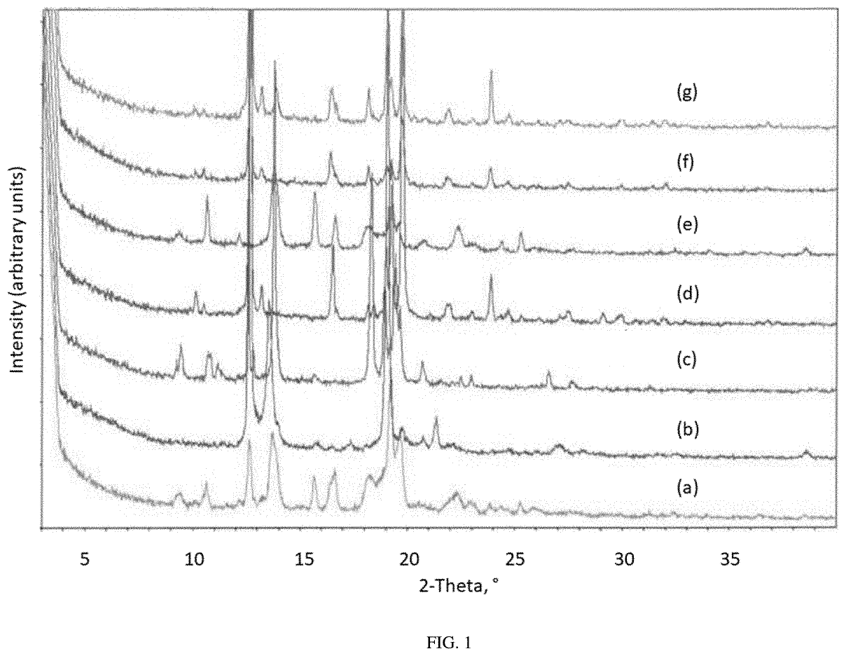

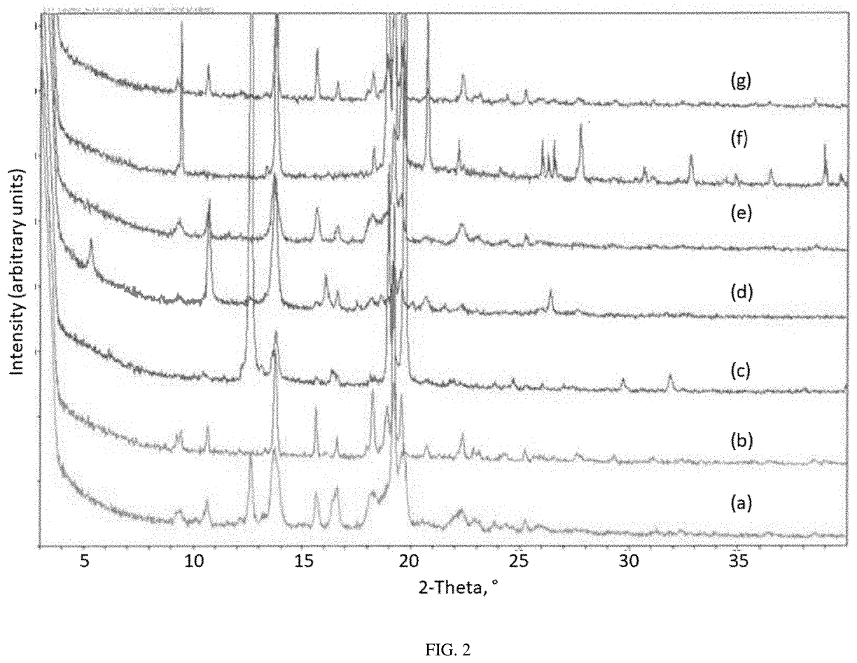

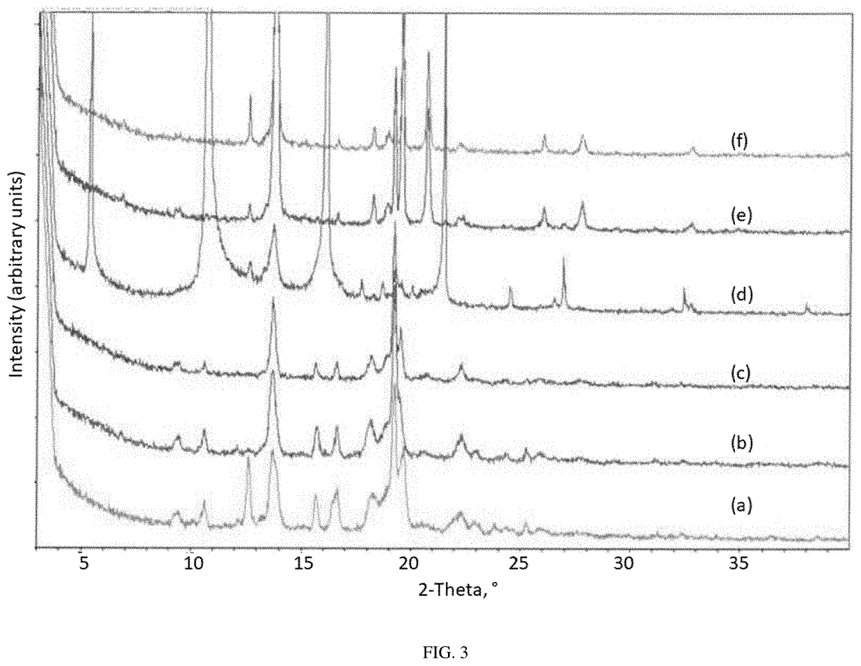

XRPD diffractograms for the solid material from various organic solvents are presented in FIGS. 1, 2, and 3. For comparison, FIGS. 1(a), 2(a), and 3(a) show the original material before exposure to the various organic solvents. An XRPD diffractogram is not available for the sample obtained from treatment with DMF. The original Example 1 compound is assigned as polymorphs “A”. Polymorphs “B” through “I” were assigned based on commonality among diffractograms from different solvents.

TABLE 4
Polymorphism of the Example 1 compound from various organic solvents

| | Observation | | Dry | | | XRPD |
|---|---|---|---|---|---|---|
| Solvent | RT | 50° C. | method | Appearance | FIGS. | pattern |
| MeOH | c | N/A | II | white powder | 1(b) | E |
| EtOH | s | c | II | pale yellow plate | 1(c) | F |
| iPrOH | c | N/A | II | white powder | 1(d) | C |
| CH₃CN | s | c | II | pale yellow powder | 1(e) | B |
| acetone | c | c | II | white powder | 1(f) | C |
| MEK | c | N/A | II | white powder | 1(g) | C |
| MTBE | s | s | I | white powder | 2(b) | B |
| EtOAc | c | N/A | II | white powder | 2(c) | C |
| THF | c | N/A | II | white powder | 2(d) | G |
| CH₂Cl₂ | c | N/A | II | white powder | 2(e) | B |
| DMSO | c | N/A | II | white powder | 2(f) | H |
| toluene | s | s | I | white powder | 2(g) | B |
| DMF | c | N/A | II | white powder | | N/A |
| heptane | s | s | I | white powder | 3(b) | B |
| H₂O | s | s | I | white powder | 3(c) | B |
| dioxane | c | N/A | II | damp solid | 3(d) | I |
| 1/1 (v/v) | s | s | I | white powder | 3(e) | D |
| EtOH/H₂O | | | | | | |
| 1/1 (v/v) | s | s | I | white powder | 3(f) | D |
| acetone/H₂O | | | | | | |

“s” = slurry; “c” = clear

XRPD diffractograms were recorded for polymorphs “A” through “I”. The 25 strongest peaks from each polymorph are reported in Tables 5 through Table 13, below.

TABLE 5
Polymorph “A” of the Example 1 compound.

| | | relative |
|---|---|---|
| 2-Theta, ° | d-spacing, Å | height |
| 3.54 | 24.93 | 25.3 |
| 9.45 | 9.35 | 7.9 |
| 10.71 | 8.25 | 13.6 |
| 12.21 | 7.24 | 3.8 |
| 13.77 | 6.43 | 62.6 |
| 15.72 | 5.63 | 23.8 |
| 16.67 | 5.31 | 18.4 |
| 18.23 | 4.86 | 27.3 |
| 19.25 | 4.61 | 100.0 |
| 19.61 | 4.52 | 39.9 |
| 20.75 | 4.28 | 4.2 |
| 22.37 | 3.97 | 16.7 |
| 23.10 | 3.85 | 4.0 |
| 24.44 | 3.64 | 5.2 |
| 25.31 | 3.52 | 7.2 |
| 25.98 | 3.43 | 4.5 |
| 27.75 | 3.21 | 2.9 |
| 29.43 | 3.03 | 1.7 |
| 31.11 | 2.87 | 1.9 |
| 32.45 | 2.76 | 2.8 |
| 32.81 | 2.73 | 1.3 |
| 34.06 | 2.63 | 1.7 |
| 35.82 | 2.50 | 2.2 |
| 36.51 | 2.46 | 1.4 |
| 38.56 | 2.33 | 3.4 |

TABLE 6
Polymorph “B” of the Example 1 compound.

| | | relative |
|---|---|---|
| 2-Theta, ° | d-spacing, Å | height |
| 3.52 | 25.07 | 100.0 |
| 9.37 | 9.43 | 6.6 |
| 10.67 | 8.28 | 29.4 |
| 12.17 | 7.27 | 7.4 |
| 13.79 | 6.42 | 49.8 |
| 15.70 | 5.64 | 33.4 |
| 16.65 | 5.32 | 20.3 |
| 18.13 | 4.89 | 8.8 |
| 19.23 | 4.61 | 49.5 |
| 19.60 | 4.52 | 12.7 |
| 20.83 | 4.26 | 5.5 |
| 22.33 | 3.98 | 13.7 |
| 23.17 | 3.84 | 3.6 |
| 24.37 | 3.65 | 6.7 |
| 25.30 | 3.52 | 12.3 |
| 25.92 | 3.43 | 2.8 |
| 32.46 | 2.76 | 3.6 |
| 34.08 | 2.63 | 2.5 |
| 38.56 | 2.33 | 4.4 |

TABLE 7
Polymorph “C” of the Example 1 compound.

| | | relative |
|---|---|---|
| 2-Theta, ° | d-spacing, Å | height |
| 3.31 | 26.71 | 35.3 |
| 3.54 | 24.93 | 20.5 |
| 9.90 | 8.92 | 0.8 |
| 10.16 | 8.70 | 2.8 |
| 10.53 | 8.39 | 1.3 |
| 12.30 | 7.19 | 0.8 |
| 12.65 | 6.99 | 100.0 |
| 13.20 | 6.70 | 3.6 |
| 16.55 | 5.35 | 10.0 |
| 18.20 | 4.87 | 1.8 |
| 18.46 | 4.80 | 1.7 |
| 19.02 | 4.66 | 10.5 |
| 19.27 | 4.60 | 17.2 |
| 19.76 | 4.49 | 49.4 |
| 21.08 | 4.21 | 0.7 |
| 21.99 | 4.04 | 2.0 |
| 22.99 | 3.87 | 0.9 |
| 23.91 | 3.72 | 6.0 |
| 24.37 | 3.65 | 0.7 |
| 24.72 | 3.60 | 1.4 |
| 27.12 | 3.29 | 1.0 |
| 27.48 | 3.24 | 1.4 |
| 29.08 | 3.07 | 1.2 |
| 29.98 | 2.98 | 1.2 |
| 30.65 | 2.91 | 0.6 |

TABLE 8
Polymorph “D” of the Example 1 compound.

| | | relative |
|---|---|---|
| 2-Theta, ° | d-spacing, Å | height |
| 3.50 | 25.21 | 17.8 |
| 9.28 | 9.52 | 0.7 |
| 12.64 | 7.00 | 1.5 |
| 13.79 | 6.42 | 100.0 |
| 18.25 | 4.86 | 2.5 |
| 18.90 | 4.69 | 1.8 |
| 19.21 | 4.62 | 14.5 |
| 19.56 | 4.53 | 26.8 |
| 20.71 | 4.28 | 10.8 |
| 22.14 | 4.01 | 0.9 |
| 22.37 | 3.97 | 1.0 |
| 26.02 | 3.42 | 2.0 |
| 26.96 | 3.30 | 0.5 |
| 27.75 | 3.21 | 2.6 |
| 29.39 | 3.04 | 0.4 |
| 31.07 | 2.88 | 0.5 |
| 32.78 | 2.73 | 0.9 |
| 34.88 | 2.57 | 0.4 |

TABLE 9
Polymorph “E” of the Example 1 compound.

| | | relative |
|---|---|---|
| 2-Theta, ° | d-spacing, Å | height |
| 3.52 | 25.07 | 19.0 |
| 12.67 | 6.98 | 100.0 |
| 13.55 | 6.53 | 16.4 |
| 14.02 | 6.31 | 2.5 |
| 15.89 | 5.57 | 0.9 |
| 16.53 | 5.36 | 0.6 |
| 17.40 | 5.09 | 1.2 |
| 19.07 | 4.65 | 52.1 |
| 19.78 | 4.48 | 1.5 |
| 20.77 | 4.27 | 1.0 |
| 21.36 | 4.16 | 3.5 |
| 22.15 | 4.01 | 0.7 |
| 24.75 | 3.59 | 0.6 |
| 26.11 | 3.41 | 0.5 |
| 26.94 | 3.31 | 1.0 |
| 27.16 | 3.28 | 1.2 |
| 28.29 | 3.15 | 0.5 |
| 38.62 | 2.33 | 0.9 |

TABLE 10
Polymorph “F” of the Example 1 compound.

| | | relative |
|---|---|---|
| 2-Theta, ° | d-spacing, Å | height |
| 3.56 | 24.79 | 33.5 |
| 9.47 | 9.33 | 7.3 |
| 10.73 | 8.24 | 5.8 |
| 11.18 | 7.91 | 3.5 |
| 12.59 | 7.02 | 100.0 |
| 12.85 | 6.89 | 5.9 |
| 13.81 | 6.41 | 70.2 |
| 15.12 | 5.86 | 1.3 |
| 15.70 | 5.64 | 1.8 |
| 18.34 | 4.83 | 44.6 |
| 18.97 | 4.67 | 20.7 |
| 19.47 | 4.56 | 27.2 |
| 19.67 | 4.51 | 15.9 |
| 20.72 | 4.28 | 4.8 |
| 21.55 | 4.12 | 1.2 |
| 22.49 | 3.95 | 1.9 |
| 22.98 | 3.87 | 2.6 |
| 26.26 | 3.39 | 1.2 |
| 26.63 | 3.35 | 3.9 |
| 27.67 | 3.22 | 1.8 |
| 31.29 | 2.86 | 0.9 |
| 38.76 | 2.32 | 0.8 |
| 39.11 | 2.30 | 0.8 |

TABLE 11
Polymorph “G” of the Example 1 compound.

| | | relative |
|---|---|---|
| 2-Theta, ° | d-spacing, Å | height |
| 3.52 | 25.07 | 100.0 |
| 5.37 | 16.44 | 16.1 |
| 10.73 | 8.24 | 55.9 |
| 12.60 | 7.02 | 3.1 |
| 13.77 | 6.43 | 67.5 |
| 15.71 | 5.63 | 2.9 |
| 16.11 | 5.50 | 18.1 |
| 16.67 | 5.32 | 9.2 |
| 18.26 | 4.86 | 4.1 |
| 18.66 | 4.75 | 4.7 |
| 19.23 | 4.61 | 33.8 |
| 19.53 | 4.54 | 18.5 |
| 20.71 | 4.29 | 6.8 |
| 21.58 | 4.11 | 3.5 |
| 22.39 | 3.97 | 4.7 |
| 23.02 | 3.86 | 2.4 |
| 26.09 | 3.41 | 3.3 |
| 26.41 | 3.37 | 13.0 |
| 27.65 | 3.22 | 2.7 |

TABLE 12
Polymorph “H” of the Example 1 compound.

| | | relative |
|---|---|---|
| 2-Theta, ° | d-spacing, Å | height |
| 3.21 | 27.53 | 20.1 |
| 3.52 | 25.07 | 11.2 |
| 9.47 | 9.33 | 9.7 |
| 13.38 | 6.61 | 0.8 |
| 13.81 | 6.41 | 55.2 |
| 17.69 | 5.01 | 0.4 |
| 18.94 | 4.68 | 61.6 |
| 19.24 | 4.61 | 34.6 |
| 19.61 | 4.52 | 100.0 |
| 20.75 | 4.28 | 12.4 |
| 22.17 | 4.01 | 2.0 |
| 22.39 | 3.97 | 0.5 |
| 24.08 | 3.69 | 0.6 |
| 25.30 | 3.52 | 0.4 |
| 26.02 | 3.42 | 2.5 |
| 26.30 | 3.39 | 1.6 |
| 26.57 | 3.35 | 2.5 |
| 27.77 | 3.21 | 3.5 |
| 30.67 | 2.91 | 1.0 |
| 31.05 | 2.88 | 0.3 |
| 32.80 | 2.73 | 2.0 |
| 34.89 | 2.57 | 0.7 |
| 36.51 | 2.46 | 1.0 |
| 38.96 | 2.31 | 2.6 |
| 39.69 | 2.27 | 0.8 |

TABLE 13
Polymorph “I” of the Example 1 compound.

| | d-spacing, | relative |
|---|---|---|
| 2-Theta, ° | Å | height |
| 3.31 | 26.71 | 13.0 |
| 3.56 | 24.80 | 7.7 |
| 5.42 | 16.28 | 16.5 |
| 10.75 | 8.22 | 100.0 |
| 12.68 | 6.97 | 0.7 |
| 13.32 | 6.64 | 0.7 |
| 13.71 | 6.45 | 2.9 |
| 16.10 | 5.50 | 54.5 |
| 17.73 | 5.00 | 0.9 |
| 19.27 | 4.60 | 1.7 |
| 19.57 | 4.53 | 0.9 |
| 20.04 | 4.43 | 0.6 |
| 21.50 | 4.13 | 16.3 |
| 23.69 | 3.75 | 0.2 |
| 24.51 | 3.63 | 1.1 |
| 26.53 | 3.36 | 0.5 |
| 26.92 | 3.31 | 2.6 |
| 32.43 | 2.76 | 1.3 |
| 32.76 | 2.73 | 0.4 |
| 38.01 | 2.37 | 0.4 |

DSC studies were performed for the Example 1 compound (polymorph A), and for the materials obtained from CH₃CN (polymorph B), iPrOH (polymorph C), and 1/1 (v/v) EtOH/H₂O (polymorph D). Results are provided in Table 14.

TABLE 14
DSC of polymorphs of the Example 1 compound

| | | endotherm | energy | exotherm | energy |
|---|---|---|---|---|---|
| | XRPD | onset, | absorbed, | onset, | released, |
| solvent | pattern | ° C. | J/g | ° C. | J/g |
| N/A | A | 238.83 | 71.67 | 240.99 | 628.3 |
| CH₃CN | B | 236.80 | 78.97 | 241.08 | 582.8 |
| iPrOH | C | 238.20 | 79.94 | 241.62 | 601.6 |
| 1/1 (v/v) | D | 243.62 | 99.87 | 247.01 | 658.2 |
| EtOH/H₂O | | | | | |

### Preliminary Salt Formation Experiments with the Example 1 Compound

The following general procedure was used to conduct salt experiments with Example 1. About 20 mg of the Example 1 compound was weighed into individual vials. For salts of liquid acids, 500 μL of solvent was added to an individual vial and stirred at 50° C. (700 rpm) to dissolve the free base, then a solution of the liquid acid (0.5 M, diluted by corresponding solvents) with fixed molar ratio (1:1.1) was added, and the mixture was stirred at 50° C. for 4 hours (700 rpm). For salts of solid acids, a quantity of the solid acid (1.1 eq relative to the free base) was added to an individual vial containing the free base as a solid, then 500 aL of a solvent was added to the vial. The resulting mixture was stirred at 50° C. (700 rpm) for 4 hours. After that, the mixture was cooled to room temperature overnight. If a solid precipitate was present, the solid precipitate was then removed by centrifugation and dried under vacuum (Method I). If a solid precipitate was not present, the solvent was removed by evaporation and the resulting material was dried under vacuum at 30° C. overnight (Method II). In either case, the resulting material was evaluated by XRPD. Control samples were also processed by using same procedure as above. DSC testing were carried out on the solid material that formed.

TABLE 15
Summary of salt experiments.

| | | Recrystallization solvent | | | |
|---|---|---|---|---|---|
| | Salt | | | | 95% iPrOH/ |
| Acid | Ex. | Acetone | EtOAc | CH₃CN | 5% H₂O |
| HCl | 2 | Free base | Crystal | Free base | Free base |
| H₂SO₄ | 3 | Crystal | Amorphous | Amorphous | Crystal |
| H₃PO₄ | 4 | Amorphous | Amorphous | Free base | Free base |
| Methane- | 5 | Crystal | Free base | Crystal | Crystal |
| sulfonic acid | | | | | |
| Tartaric acid | 6 | Amorphous | Crystal | Free base | Free base |
| L-Malic acid | 7 | Crystal | Crystal | Crystal | Crystal |
| Citric acid (1) | 8 | Crystal | Crystal | Crystal | Crystal |
| Maleic acid | 9 | Crystal | Crystal | Crystal | Crystal |
| Succinic acid | 10 | Crystal | Crystal | Crystal | Crystal |
| Fumaric acid | 11 | Crystal | Crystal | Crystal | Crystal |

(1) Citric acid was supplied as its monohydrate.

The crystallization process was observed. Appearance of the various crystallization procedures is summarized in Tables 16 and 17.

TABLE 16
Observation of salt formation experiments (liquid acids).

| | | Solvent | | | |
|---|---|---|---|---|---|
| | Salt | | | | 95% iPrOH/ |
| Acid | Ex. | Acetone | EtOAc | CH₃CN | 5% H₂O |
| HCl | 2 | c-c-c-c | c-s-s-s | s-c-c-c | c-c-c-c |
| H₂SO₄ | 3 | c-s-s-s | c-s-s-s | s-s-s-s | c-s-s-s |
| H₃PO₄ | 4 | c-s-s-s | c-s-s-s | s-s-s-s | c-s-s-s |
| Methane- | 5 | c-c-s-s | c-c-c-c | s-c-c-s | c-c-s-s |
| sulfonic acid | | | | | |

Before adding acid - After adding acid - After 4 hrs stirring - After overnight cooling
“s” = suspension; “c” = clear

TABLE 17
Observation of salt formation experiments (solid acids).

| | | Solvent | | | |
|---|---|---|---|---|---|
| | Salt | | | | 95% iPrOH/ |
| Acid | Ex. | Acetone | EtOAc | CH₃CN | 5% H₂O |
| Tartaric acid | 6 | c-c-c | s-s-s | s-c-s | c-c-c |
| L-Malic acid | 7 | c-c-c | s-c-s | s-s-s | c-c-c |
| Citric acid (1) | 8 | c-c-c | s-s-s | s-c-s | c-c-c |
| Maleic acid | 9 | s-s-s | s-s-s | s-s-s | s-s-s |
| Succinic acid | 10 | c-c-c | s-s-s | s-s-s | c-c-c |
| Fumaric acid | 11 | s-s-s | s-s-s | s-s-s | c-s-s |

(1) Citric acid was supplied as its monohydrate.
After adding solvent - After 4 hrs stirring - After overnight cooling
“s” = suspension; “c” = clear

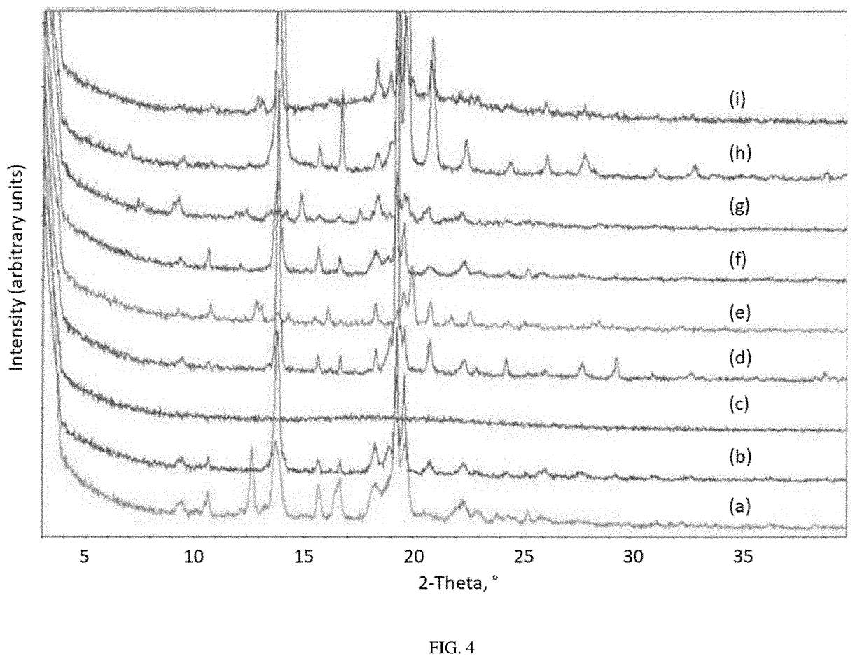

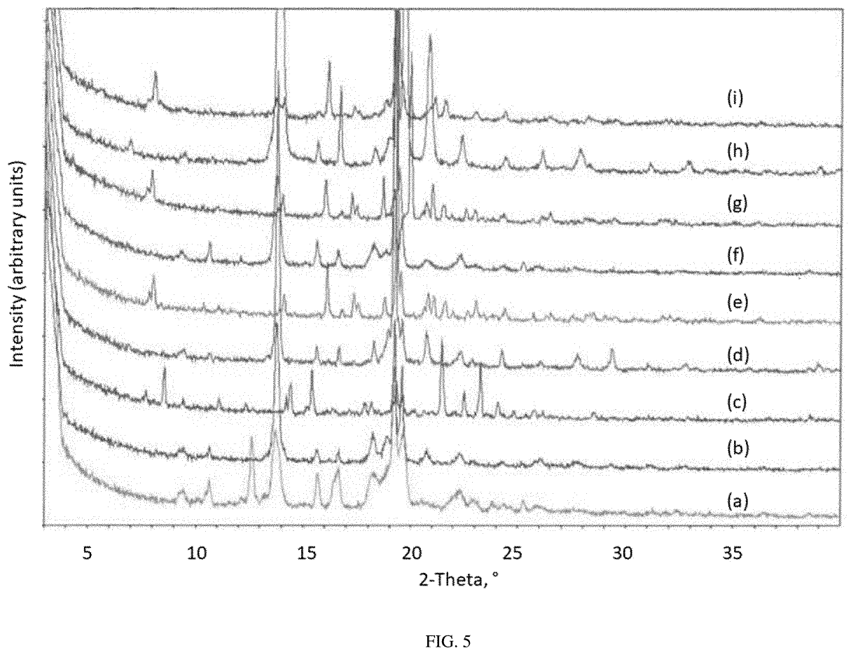

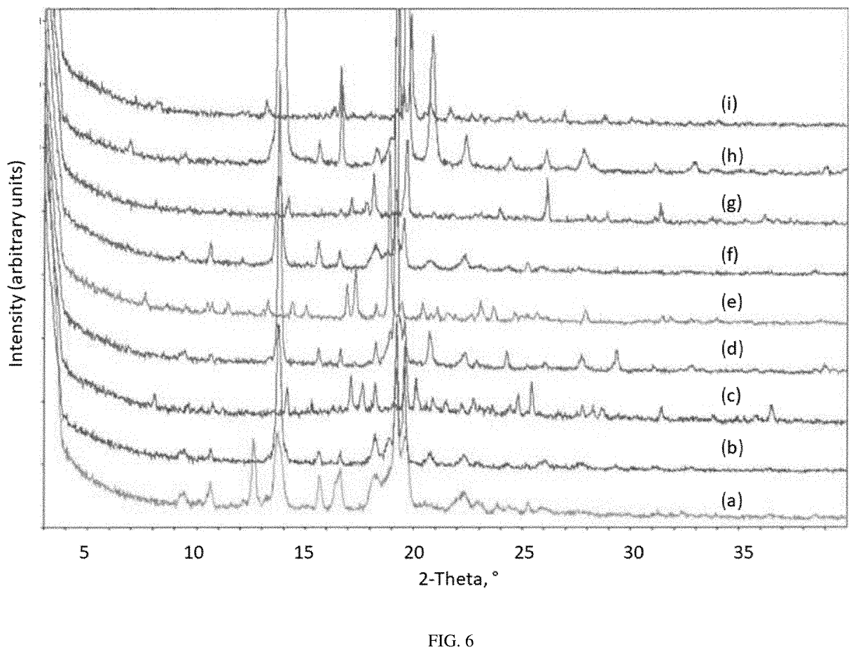

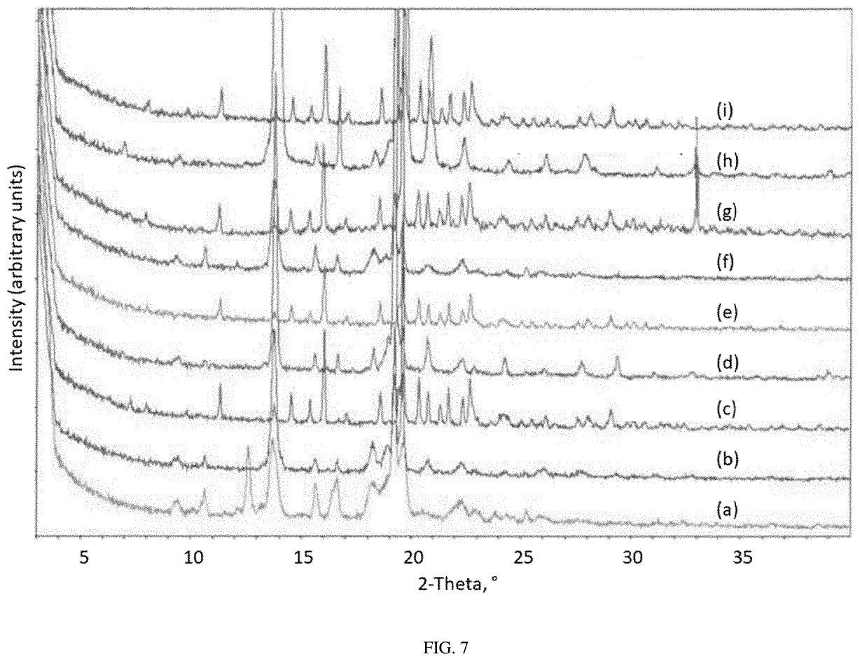

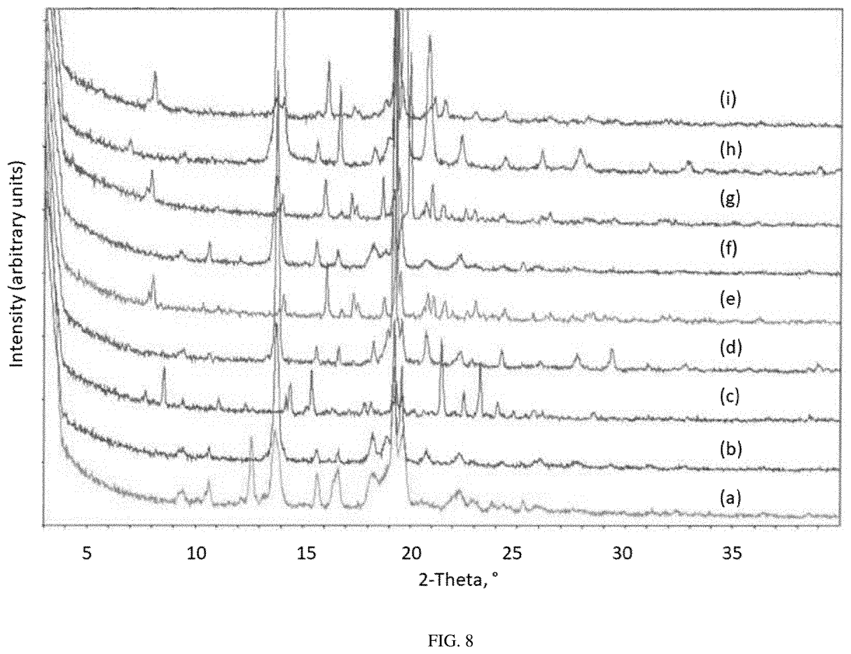

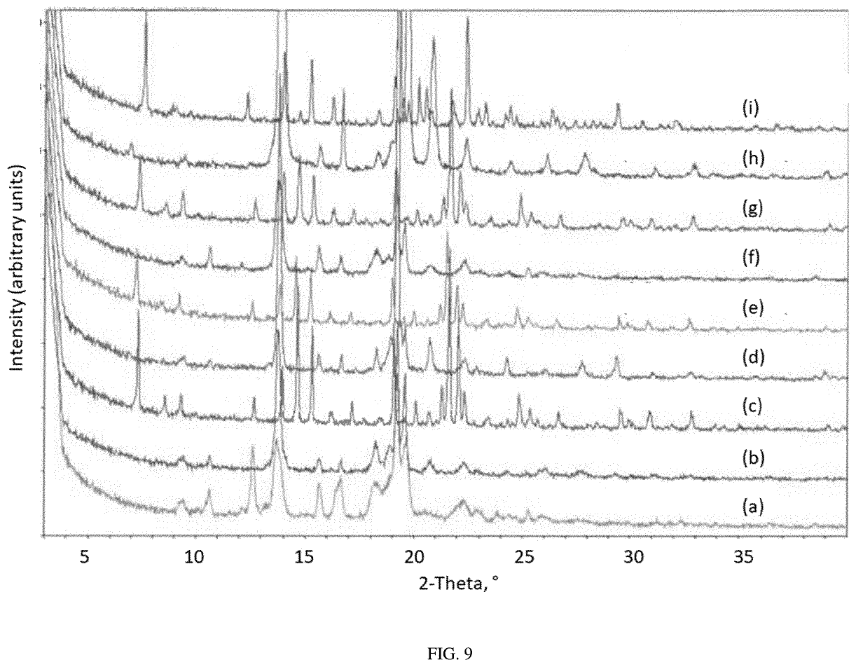

XRPD diffractograms for the salt formation experiments with solid acids are presented in FIGS. 4 through 9, and are summarized in Table 18. For comparison, trace (a) in each of FIGS. 4 through 9 show the Example 1 compound. Traces (b), (d), (f), and (h) in each of FIGS. 4 through 9 show a control of the Example 1 compound from the indicated solvent, in the absence of acid. Traces (c), (e), (g), and (i) in each of FIGS. 4 through 9 show the product from combination of the Example 1 compound and the indicated acid in the indicated solvent. Method I denotes combinations for which a solid was isolated by centrifugation, as described above. Method II denotes combinations for which a solid was isolated by evaporation, as described above.

TABLE 18
XRPD diffractograms of salt formation experiments from solid acids.

| | | solvent | | | |
|---|---|---|---|---|---|
| | | | | | 95% iPrOH/ |
| | | acetone | EtOAc | ACN | 5% H₂O |
| acid | contents | FIG/method | FIG/method | FIG/method | FIG/method |
| tartaric | 1 | 4(b)/II | 4(d)/II | 4(f)/I | 4(h)/II |
| | 1 + acid | 4(c)/II | 4(e)/I | 4(g)/II | 4(i)/II |
| L-malic | 1 | 5(b)/II | 5(d)/II | 5(f)/II | 5(h)/II |
| | 1 + acid | 5(c)/II | 5(e)/II | 5(g)/I | 5(i)/II |
| citric | 1 | 6(b)/II | 6(d)/II | 6(f)/I | 6(h)/II |
| | 1 + acid | 6(c)/II | 6(e)/I | 6(g)/II | 6(i)/II |
| maleic | 1 | 7(b)/II | 7(d)/II | 7(f)/I | 7(h)/II |
| | 1 + acid | 7(c)/I | 7(e)/I | 7(g)/I | 7(i)/I |
| succinic | 1 | 8(b)/II | 8(d)/II | 8(f)/I | 8(h)/II |
| | 1 + acid | 8(c)/II | 8(e)/I | 8(g)/I | 8 (i)/II |
| fumaric | 1 | 9(b)/II | 9(d)/II | 9(f)/I | 9(h)/II |
| | 1 + acid | 9(c)/I | 9(e)/I | 9(g)/I | 9(i)/I |

TABLE 19
XRPD of hydrogen fumarate salt 11 from
preliminary salt formation experiment.

| | d-spacing, | relative |
|---|---|---|
| 2-Theta, ° | Å | height |
| 3.34 | 26.40 | 90.8 |
| 3.72 | 23.74 | 41.7 |
| 7.34 | 12.03 | 56.0 |
| 8.59 | 10.29 | 10.1 |
| 9.35 | 9.46 | 12.0 |
| 12.72 | 6.96 | 13.4 |
| 13.97 | 6.33 | 31.7 |
| 14.68 | 6.03 | 76.8 |
| 15.35 | 5.77 | 54.7 |
| 16.23 | 5.46 | 6.4 |
| 17.20 | 5.15 | 12.4 |
| 19.12 | 4.64 | 48.3 |
| 20.10 | 4.41 | 13.2 |
| 20.75 | 4.28 | 7.2 |
| 21.32 | 4.16 | 21.3 |
| 21.64 | 4.10 | 100.0 |
| 22.06 | 4.03 | 61.2 |
| 22.34 | 3.98 | 18.1 |
| 24.84 | 3.58 | 18.5 |
| 25.35 | 3.51 | 10.4 |
| 26.68 | 3.34 | 9.1 |
| 29.55 | 3.02 | 10.8 |
| 29.95 | 2.98 | 5.9 |
| 30.91 | 2.89 | 11.3 |
| 32.84 | 2.72 | 10.9 |

DSC were performed for the HCl, H₂SO₄, and methanesulfonic acid salts 2, 3, and 5, respectively (data not shown). Weight loss of 10.82%, 4.77%, and 2.15% were observed, respectively. In no case was a clean phase transition indicated in the DSC.

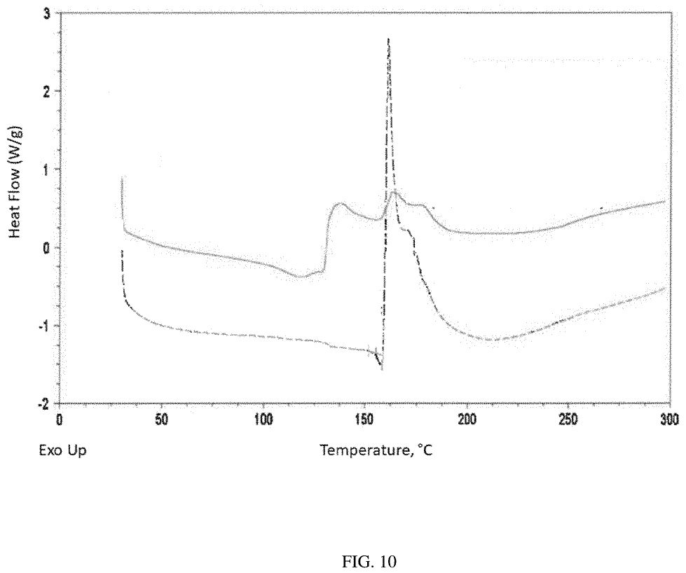

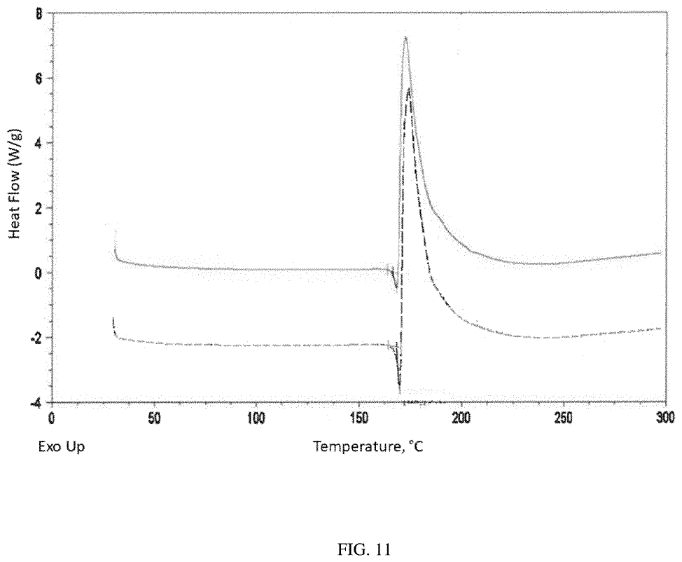

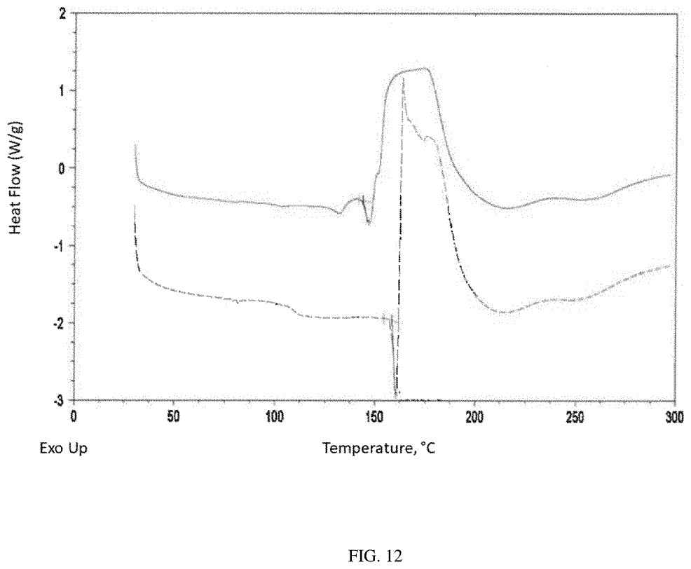

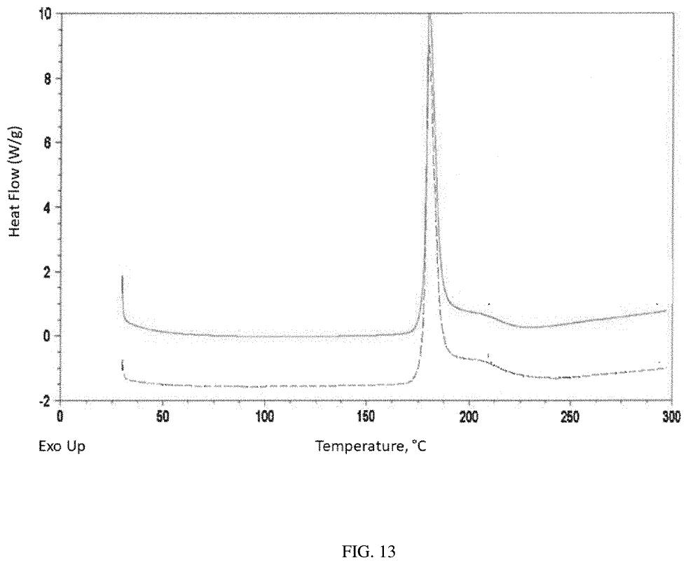

DSC were performed for the hydrogen tartrate, hydrogen L-malate, dihydrogen citrate, hydrogen maleate, hydrogen succinate, and hydrogen fumarate salts. Both the hydrogen tartrate salt 6 and the hydrogen L-malate salt 7 displayed broad transitions in the DSC over a wide temperature range (data not shown). Traces for the dihydrogen citrate, hydrogen maleate, hydrogen succinate, and hydrogen fumarate salts (8, 9, 10, and 11, respectively) are shown in FIGS. 10, 11, 12, and 13, respectively.

### Scale-Up (100 mg) for Salts of the Example 1 Compound

Scale-up was performed for the Example 1 compound for each of the acids from the preliminary salt formation experiments, except for the phosphoric acid salt 4, which formed amorphous material from both acetone and EtOAc.

For liquid acids, individual vials were prepared with 2.5 mL of the indicated solvent and 100 mg of the Example 1 compound, and the mixtures were stirred at 50° C. (700 rpm) to dissolution. Solutions of each selected acid in the indicated solvent (0.5 M) with fixed molar ratio (1:1.1 Example 1 compound:acid) were then added to individual vials containing the solutions of the Example 1 compound. The resulting solutions were stirred at 50° C. for 4 hours (700 rpm). Visual observation of reaction progress is reported in Table 20.

TABLE 20
Scale-up of salt preparation (liquid acids)

| | Salt | | | mass loss | |
|---|---|---|---|---|---|
| Acid | Ex. | solvent | observation | from DSC | characterization |
| HCl | 2 | EtOAc | c-s-s-s | 10.82% at | nearly amorphous |
| | | | | 19.92° C. | |
| H₂SO₄ | 3 | Acetone | c-s-s-s | 4.77% at | same XRPD as sample |
| | | | | 119.35° C. | from preliminary |
| | | | | | experiment; |
| | | | | | likely a solvate |
| Methane- | 5 | Acetone | c-c-c-p | 4.59% at | same XRPD as sample |
| sulfonic acid | | | | 119.64° C. | from preliminary |
| | | | | | experiment; |
| | | | | | likely a solvate |

Before adding acid - After adding acid - After 4 hrs stirring - After overnight cooling
“s” = suspension; “c” = clear; “p” = precipitate

For solid acids, certain amount of each selected acid (1.1 eq to free form) was added into an individual vial containing 100 mg of the solid Example 1 compound, then 2.5 mL of solvent was added, then the result solution/suspension was stirred at 50° C. (700 rpm) for 4 hours. After that, the mixture were cooled to room temperature overnight. Then the solid precipitates were centrifuged and vacuum drying, for clear solutions, solutions were vacuum dried at 30° C. directly for 4 hours to precipitate solids by evaporation, solids were collected and evaluated by XRPD. DSC and TGA testing were carried out on the solid material that formed. Visual observation of reaction progress is reported in Table 21.

TABLE 21
Observation of salt preparation (solid acids)

| | Salt | | | mass loss | |
|---|---|---|---|---|---|
| Acid | Ex. | solvent | observation | from TGA | characterization |
| Tartaric acid | 6 | EtOAc | s-s-s | 5.13% at | nearly amorphous |
| | | | | 119.64° | |
| L-Malic acid | 7 | Acetone | c-c-c | 2.15% at | amorphous |
| | | | | 119.92° C. | |
| Citric acid (1) | 8 | EtOAc | s-s-s | 0.164% at | different XRPD from |
| | | | | 119.92° C. | preliminary experiment |
| Maleic acid | 9 | EtOAc | s-s-s | 0.109% at | same XRPD as |
| | | | | 119.92° C. | preliminary experiment |
| Succinic acid | 10 | EtOAc | c-s-s | 0.112% at | same XRPD as |
| | | | | 118.64° C. | preliminary experiment |
| Fumaric acid | 11 | Acetone | s-s-s | 0.196% at | same XRPD as |
| | | | | 120° C. | preliminary experiment |

(1) Citric acid was supplied as its monohydrate.

TABLE 22
XRPD of hydrogen sulfate salt 3 from 100 mg scale-up reaction

| | d-spacing, | relative |
|---|---|---|
| 2-Theta, ° | Å | height |
| 3.31 | 26.70 | 44.0 |
| 3.71 | 23.80 | 19.2 |
| 5.96 | 14.82 | 4.6 |
| 8.03 | 11.01 | 31.8 |
| 11.23 | 7.87 | 1.5 |
| 14.83 | 5.97 | 1.3 |
| 16.06 | 5.52 | 100.0 |
| 16.77 | 5.28 | 2.8 |
| 17.88 | 4.96 | 3.7 |
| 19.02 | 4.66 | 3.8 |
| 19.21 | 4.62 | 2.1 |
| 19.85 | 4.47 | 13.0 |
| 20.61 | 4.31 | 2.7 |
| 20.89 | 4.25 | 3.2 |
| 21.78 | 4.08 | 5.1 |
| 22.93 | 3.88 | 2.8 |
| 23.83 | 3.73 | 4.3 |
| 24.15 | 3.68 | 12.7 |
| 25.63 | 3.47 | 1.3 |
| 28.01 | 3.18 | 1.1 |
| 30.67 | 2.91 | 1.2 |
| 31.84 | 2.81 | 1.5 |
| 33.58 | 2.67 | 1.1 |

TABLE 23
XRPD of methane sulfonate salt 5 from 100 mg scale-up reaction

| | d-spacing, | relative |
|---|---|---|
| 2-Theta, ° | Å | height |
| 3.36 | 26.24 | 66.5 |
| 3.64 | 24.26 | 35.4 |
| 6.87 | 12.85 | 18.2 |
| 9.96 | 8.87 | 5.6 |
| 10.28 | 8.60 | 3.2 |
| 11.22 | 7.88 | 2.6 |
| 13.77 | 6.42 | 43.7 |
| 15.51 | 5.71 | 3.0 |
| 15.74 | 5.63 | 14.4 |
| 16.52 | 5.36 | 2.9 |
| 17.17 | 5.16 | 6.4 |
| 19.13 | 4.63 | 100.0 |
| 19.55 | 4.54 | 9.2 |
| 20.69 | 4.29 | 11.3 |
| 21.42 | 4.14 | 50.9 |
| 22.94 | 3.87 | 2.4 |
| 23.61 | 3.77 | 2.3 |
| 24.36 | 3.65 | 6.1 |
| 25.29 | 3.52 | 3.7 |
| 25.98 | 3.43 | 2.3 |
| 27.67 | 3.22 | 5.0 |
| 28.01 | 3.18 | 4.5 |
| 29.51 | 3.02 | 3.4 |
| 33.35 | 2.68 | 2.5 |
| 37.46 | 2.40 | 2.6 |

TABLE 24
XRPD of hydrogen tartrate salt 6 from 100 mg scale-up reaction

| | d-spacing, | relative |
|---|---|---|
| 2-Theta, ° | Å | height |
| 3.62 | 24.39 | 100.0 |
| 20.79 | 4.27 | 25.6 |
| 7.37 | 11.98 | 17.4 |
| 10.42 | 8.49 | 15.8 |
| 20.50 | 4.33 | 13.5 |
| 19.29 | 4.60 | 13.3 |
| 32.10 | 2.79 | 12.5 |
| 25.17 | 3.54 | 11.0 |
| 13.70 | 6.46 | 9.8 |
| 19.59 | 4.53 | 9.6 |
| 22.57 | 3.94 | 8.0 |
| 29.82 | 2.99 | 8.0 |
| 29.27 | 3.05 | 7.6 |
| 17.19 | 5.15 | 7.0 |
| 26.64 | 3.34 | 6.8 |
| 35.91 | 2.50 | 6.6 |
| 36.81 | 2.44 | 6.1 |
| 33.54 | 2.67 | 5.3 |

TABLE 25
XRPD of dihydrogen citrate salt 8 from 100 mg scale-up reaction

| | d-spacing, | relative |
|---|---|---|
| 2-Theta, ° | Å | height |
| 3.29 | 26.87 | 100.0 |
| 3.64 | 24.26 | 46.7 |
| 7.84 | 11.26 | 6.1 |
| 9.61 | 9.19 | 9.6 |
| 12.43 | 7.12 | 10.5 |
| 12.72 | 6.95 | 12.0 |
| 13.36 | 6.62 | 15.9 |
| 14.69 | 6.03 | 3.6 |
| 15.11 | 5.86 | 17.3 |
| 16.98 | 5.22 | 4.1 |
| 17.22 | 5.15 | 17.4 |
| 18.45 | 4.81 | 9.2 |
| 18.94 | 4.68 | 22.2 |
| 19.27 | 4.60 | 14.6 |
| 19.61 | 4.52 | 13.9 |
| 20.26 | 4.38 | 15.4 |
| 20.75 | 4.28 | 3.6 |
| 20.84 | 4.26 | 3.7 |
| 21.21 | 4.19 | 24.7 |
| 21.55 | 4.12 | 8.3 |
| 22.37 | 3.97 | 11.1 |
| 23.23 | 3.83 | 6.9 |
| 25.98 | 3.43 | 4.8 |
| 26.89 | 3.31 | 4.8 |
| 29.34 | 3.04 | 8.6 |

TABLE 26
XRPD of hydrogen maleate salt 9 from 100 mg scale-up reaction

| | d-spacing, | relative |
|---|---|---|
| 2-Theta, ° | Å | height |
| 3.64 | 24.26 | 64.5 |
| 11.38 | 7.77 | 20.1 |
| 13.80 | 6.41 | 6.3 |
| 14.60 | 6.06 | 15.5 |
| 15.43 | 5.74 | 10.7 |
| 16.06 | 5.52 | 59.3 |
| 16.82 | 5.27 | 6.1 |
| 17.07 | 5.19 | 7.4 |
| 18.62 | 4.76 | 17.0 |
| 19.31 | 4.59 | 14.0 |
| 19.61 | 4.52 | 100.0 |
| 20.16 | 4.40 | 5.7 |
| 20.36 | 4.36 | 21.6 |
| 20.79 | 4.27 | 18.3 |
| 21.38 | 4.15 | 9.7 |
| 21.74 | 4.09 | 18.6 |
| 22.39 | 3.97 | 16.0 |
| 22.71 | 3.91 | 34.9 |
| 24.10 | 3.69 | 7.3 |
| 24.40 | 3.65 | 7.2 |
| 25.53 | 3.49 | 7.2 |
| 26.21 | 3.40 | 6.0 |
| 27.61 | 3.23 | 6.6 |
| 28.09 | 3.17 | 9.5 |
| 29.15 | 3.06 | 9.7 |

TABLE 27
XRPD of hydrogen succinate salt
10 from 100 mg scale-up reaction

| | d-spacing, | relative |
|---|---|---|
| 2-Theta, ° | Å | height |
| 7.87 | 11.22 | 5.8 |
| 11.18 | 7.91 | 2.2 |
| 16.82 | 5.27 | 3.2 |
| 17.60 | 5.04 | 4.1 |
| 18.80 | 4.72 | 6.9 |
| 21.66 | 4.10 | 5.3 |
| 22.01 | 4.03 | 1.8 |
| 22.72 | 3.91 | 2.3 |
| 23.14 | 3.84 | 6.8 |
| 23.43 | 3.79 | 1.4 |
| 24.44 | 3.64 | 3.7 |
| 25.75 | 3.46 | 2.0 |
| 26.31 | 3.38 | 2.3 |
| 26.53 | 3.36 | 2.6 |
| 27.54 | 3.24 | 1.5 |
| 28.23 | 3.16 | 2.1 |
| 28.54 | 3.13 | 2.1 |
| 29.09 | 3.07 | 1.5 |
| 29.27 | 3.05 | 1.4 |
| 29.54 | 3.02 | 1.7 |
| 30.40 | 2.94 | 1.3 |
| 31.79 | 2.81 | 1.4 |
| 32.09 | 2.79 | 1.7 |
| 32.51 | 2.75 | 1.4 |
| 36.24 | 2.48 | 1.9 |

TABLE 28
XRPD of hydrogen fumarate salt 11 from 100 mg scale-up reaction

| | | relative |
|---|---|---|
| 2-Theta, ° | d-spacing, Å | height |
| 3.29 | 26.87 | 78.3 |
| 3.64 | 24.26 | 36.5 |
| 7.36 | 12.00 | 55.9 |
| 8.62 | 10.25 | 9.5 |
| 9.35 | 9.45 | 10.5 |
| 12.73 | 6.95 | 10.8 |
| 13.99 | 6.33 | 29.6 |
| 14.70 | 6.02 | 71.6 |
| 15.37 | 5.76 | 51.4 |
| 16.24 | 5.45 | 5.5 |
| 17.24 | 5.14 | 11.5 |
| 19.13 | 4.63 | 47.3 |
| 20.14 | 4.40 | 12.5 |
| 21.36 | 4.16 | 18.2 |
| 21.68 | 4.10 | 100.0 |
| 22.08 | 4.02 | 54.7 |
| 22.39 | 3.97 | 15.8 |
| 23.49 | 3.78 | 5.8 |
| 24.86 | 3.58 | 16.6 |
| 25.41 | 3.50 | 10.7 |
| 26.73 | 3.33 | 7.7 |
| 29.62 | 3.01 | 9.5 |
| 30.02 | 2.97 | 6.4 |
| 30.97 | 2.89 | 9.0 |
| 32.87 | 2.72 | 12.5 |

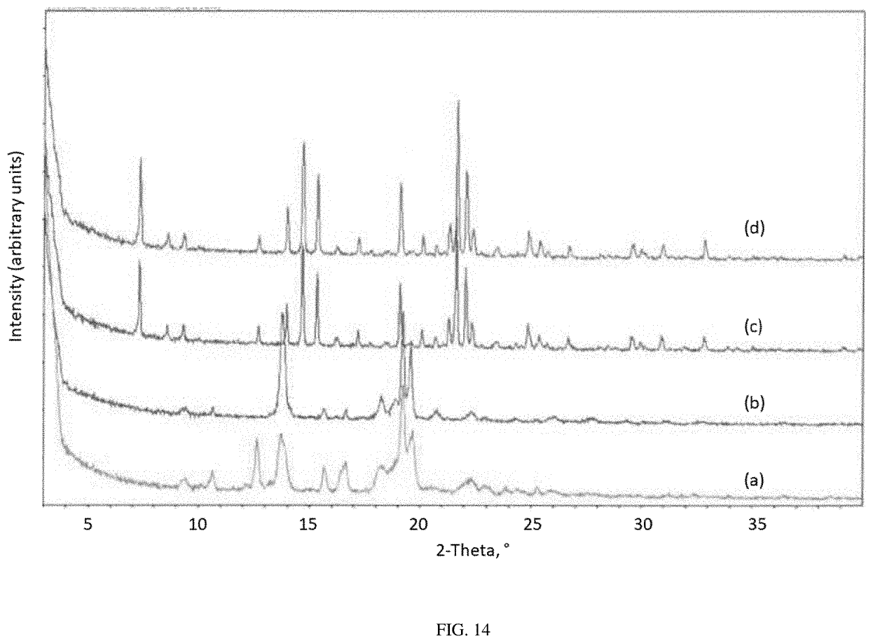

FIG. 14 provides 4 XRPD traces: (a) represents the Example 1 compound, (b) represents a control of evaporation of the Example 1 compound from acetone, (c) represents the hydrogen fumarate salt 11 from the preliminary salt formation experiment, and (d) represents the hydrogen fumarate salt 11 from the 100 mg scale-up.

The dihydrogen citrate, hydrogen maleate, hydrogen succinate, and hydrogen fumarate salts (8, 9, 10, and 11, respectively) were chosen for further study, along with the free base 1 (salts 2, 6 and 7 were substantially noncrystalline, and salts 3 and 5 were likely solvates).

TABLE 29
Summary of the properties for free base 1 and selected salts.

| | 1 | 8 | 9 | 10 | 11 |
|---|---|---|---|---|---|
| Color | white | white | white | white | white |
| Purity | 99.59% | 99.58% | 99.83% | 99.74% | 99.85% |
| Salt ratio | N/A | 1.26:1 | 1.16:1 | N/A | 1.00:1 |
| Yield | N/A | 83.21% | 82% | 63.90% | 64.84% |
| Crystallinity | Medium | Medium | Medium | Medium | High |
| Melting point | 240.33° C. | 158.52° C. | 166.74° C. | 162.62° C. | none |
| (DSC, ° C.) | | | | | |
| Enthalpy | 71.67 J/g | 9.535 J/g | 16.87 J/g | 51.14 J/g | N/A |
| (DSC, J/g) | | | | | |
| Weight loss | 0.29% | 0.19% | 0.11% | 0.11% | 0.20% |
| (TGA, %, <150° C.) | | | | | |
| Hygroscopicity | 0.11/2.44 | 0.17/0.60 | 0.07/0.27 | 0.05/0.30 | 0.10/0.47 |
| (0-40%/0-90% RH); | no change | no change | no change | no change | no change |
| form change | | | | | |

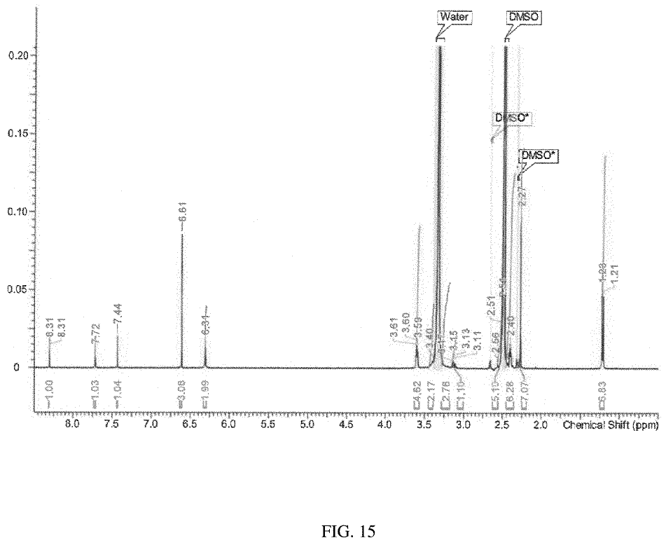

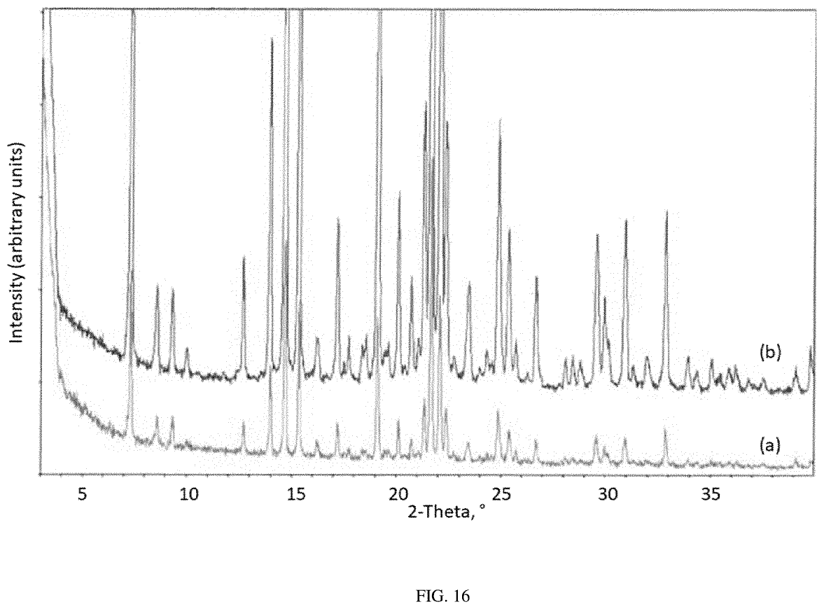

DVS behavior was recorded for the dihydrogen citrate, hydrogen maleate, hydrogen succinate, and hydrogen fumarate salts (8, 9, 10, and 11, respectively), and is reported in Tables 30, 31, 32, and 33, respectively. Total cycling time varied, but in all cases was about 200-250 min. Maximum sorption was 0.60%, 0.27%, 0.30%, and 0.47%, for 8, 9, 10, and 11, respectively. FIG. 15 shows the ¹H NMR of the hydrogen fumarate salt 11 in DMSO-d₆. FIG. 16 provides the XRPD patterns for hydrogen fumarate salt 11 before (a) and after (b) DVS.

TABLE 30
Summary of DVS behavior of dihydrogen citrate salt 8.

| | Change in mass (%) | | |
|---|---|---|---|
| Target % P/P₀ | Sorption | Desorption | Hysteresis |
| 0.0 | 0.0000 | 0.0373 | |
| 10.0 | 0.0487 | 0.0913 | 0.0426 |
| 20.0 | 0.0882 | 0.1293 | 0.0411 |
| 30.0 | 0.1255 | 0.1757 | 0.0502 |
| 40.0 | 0.1727 | 0.2145 | 0.0418 |
| 50.0 | 0.2191 | 0.2609 | 0.0418 |
| 60.0 | 0.2731 | 0.3119 | 0.0388 |
| 70.0 | 0.3423 | 0.3743 | 0.0320 |
| 80.0 | 0.4336 | 0.4565 | 0.0228 |
| 90.0 | 0.6040 | 0.6040 | |

TABLE 31
Summary of DVS behavior of hydrogen maleate salt 9.

| | Change in mass (%) | | |
|---|---|---|---|
| Target % P/P₀ | Sorption | Desorption | Hysteresis |
| 0.0 | 0.0000 | −0.0030 | |
| 10.0 | 0.0181 | 0.0196 | 0.0015 |
| 20.0 | 0.0317 | 0.0354 | 0.0038 |
| 30.0 | 0.0422 | 0.0573 | 0.0151 |
| 40.0 | 0.0656 | 0.0747 | 0.0090 |
| 50.0 | 0.0814 | 0.0950 | 0.0136 |
| 60.0 | 0.1041 | 0.1199 | 0.0158 |
| 70.0 | 0.1327 | 0.1516 | 0.0189 |
| 80.0 | 0.1787 | 0.1938 | 0.0151 |
| 90.0 | 0.2655 | 0.2655 | |

TABLE 32
Summary of DVS behavior of hydrogen succinate salt 10.

| | Change in mass (%) | | |
|---|---|---|---|
| Target % P/P₀ | Sorption | Desorption | Hysteresis |
| 0.0 | 0.0000 | 0.0514 | |
| 10.0 | 0.0151 | 0.0647 | 0.0496 |
| 20.0 | 0.0239 | 0.0727 | 0.0487 |
| 30.0 | 0.0319 | 0.0930 | 0.0611 |
| 40.0 | 0.0532 | 0.1081 | 0.0549 |
| 50.0 | 0.0727 | 0.1267 | 0.0541 |
| 60.0 | 0.0975 | 0.1489 | 0.0514 |
| 70.0 | 0.1267 | 0.1790 | 0.0523 |
| 80.0 | 0.1941 | 0.2233 | 0.0292 |
| 90.0 | 0.3013 | 0.3013 | |

TABLE 33
Summary of DVS behavior of hydrogen fumarate salt 11.

| | Change in mass (%) | | |
|---|---|---|---|
| Target % P/P₀ | Sorption | Desorption | Hysteresis |
| 0.0 | 0.0000 | −0.0114 | |
| 10.0 | 0.0200 | 0.0209 | 0.0010 |
| 20.0 | 0.0381 | 0.0448 | 0.0067 |
| 30.0 | 0.0581 | 0.0866 | 0.0286 |
| 40.0 | 0.0971 | 0.1238 | 0.0267 |
| 50.0 | 0.1343 | 0.1695 | 0.0352 |
| 60.0 | 0.1828 | 0.2285 | 0.0457 |
| 70.0 | 0.2447 | 0.2885 | 0.0438 |
| 80.0 | 0.3295 | 0.3685 | 0.0390 |
| 90.0 | 0.4742 | 0.4742 | |

Solubility of the free base 1 and the selected salts 8, 9, 10, and 11 in physiologically relevant solutions were examined. For each material, aliquots were added to 2 ml vials containing 500 uL of either SGF, FaSSIF, or FeSSIF, which were then sealed. The mixtures were kept shaking by thermomixer at 700 rpm, 25±0.5° C. for 24 h to facilitate the dissolution and then centrifuged by 96-well plates at 3000 rpm for 2 min. The filter liquors were diluted with diluents (ACN/water, 50/50) and analyzed by HPLC. Observed solubilities are presented in Table 34.

TABLE 34
Solubility of free base 1 and selected salts under physiological conditions

| | SGF | | FaSSIF | | FeSSIF | |
|---|---|---|---|---|---|---|
| | Conc | pH | Conc | pH | Conc | pH |
| Material | (ug/mL) | after 24 h | (ug/mL) | after 24 h | (ug/mL) | after 24 h |
| 1 | 4560.95 | 3.83 | 71.27 | 6.48 | 473.26 | 5.00 |
| 8 | 6331.75 | 3.26 | 113.31 | 5.53 | 772.94 | 4.86 |
| 9 | 13362.20 | 3.26 | 112.94 | 5.65 | 565.48 | 4.90 |
| 10 | 7075.30 | 3.71 | 72.24 | 5.92 | 642.30 | 4.88 |
| 11 | 5808.45 | 3.01 | 458.52 | 4.85 | 646.12 | 4.84 |

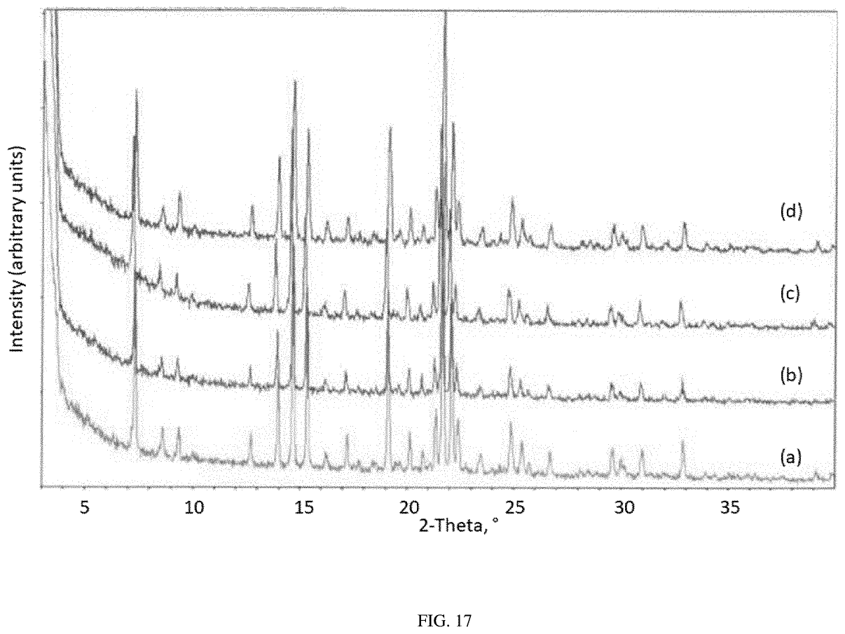

The stability of the free base 1 and the selected salts 8, 9, 10, and 11 was evaluated. For each material, aliquots were set up at each of conditions (50° C., 40° C./75% RH, light) at given time points (initial and 1 week). PXRD diffractograms of the hydrogen fumarate salt 11 is shown in FIG. 17, with trace (a) showing the initial salt, trace (b) showing the salt after light treatment, trace (c) showing the salt after storage at 40° C. and 75% relative humidity for 1 week, and (d) showing the salt after storage at 50° C. for 1 week.

TABLE 35
Stability of free base 1 and selected salts.

| | | Test conditions | | | | | |
|---|---|---|---|---|---|---|---|
| | | 40° C., 75% RH | | 50° C. | | light | |
| | | bulk, 1 week | | bulk, 1 week | | bulk, 1 week | |
| | initial | | XRPD | | XRPD | | XRPD |
| | purity | purity | change? | purity | change? | purity | change? |
| 1 | 99.59% | 99.66% | yes | 99.41 | yes | 96.61 | yes (1) |
| 8 | 99.58% | 99.41% | no | 98.91% | no | 99.44% | no |
| 9 | 99.83% | 99.87% | no | 99.89% | no | 99.79% | no |
| 10 | 99.74% | 100% | no | 100% | no | 99.74% | no (2) |
| 11 | 99.85% | 99.88% | no | 99.89% | no | 99.89% | no |

(1) yellow powder
(2) pale yellow powder

### Scale-Up (500 mg) for Salts of the Example 1 Compound

The hydrogen maleate, hydrogen succinate, and hydrogen fumarate salts (9, 10, and 11, respectively) were selected for preparation on a 500 mg scale. Each salt was prepared from the same solvent that was used for the 100 mg scale-up. Properties for the resulting salts are listed in Table 36.

TABLE 36
Summary of salt properties: scale-up.

| | 9 | 10 | 11 |
|---|---|---|---|
| Color | white | white | white |
| Solvent | EtOAc | EtOAc | acetone |
| Yield | 85.33% | 50.14% | 66.79% |
| Melting point | 167.53° C. | 160.72° C. | none |
| (DSC, ° C.) | | | |
| Enthalpy | 14.65 J/g | 16.08 J/g | N/A |
| (DSC, J/g) | | | |
| Weight loss | 0.2515% | 0.2917% | 0.1383% |
| (TGA, %, < 150° C.) | | | |

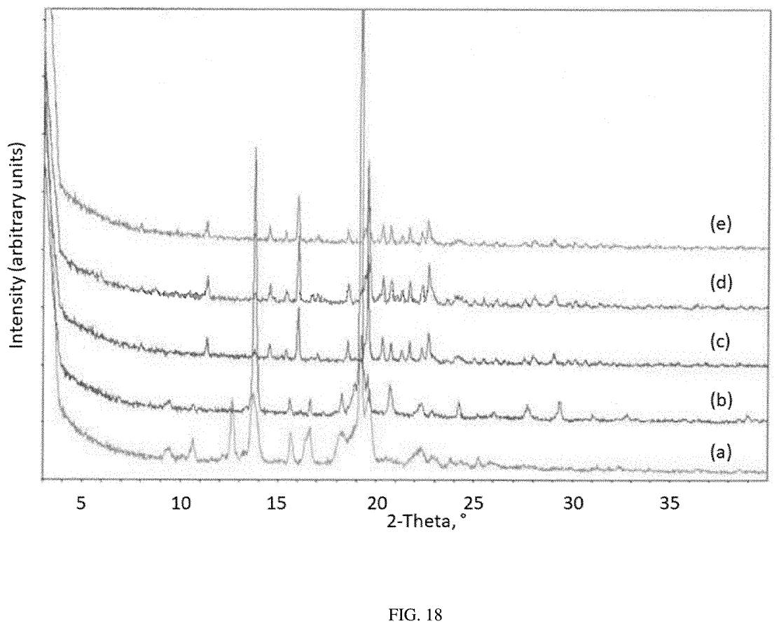

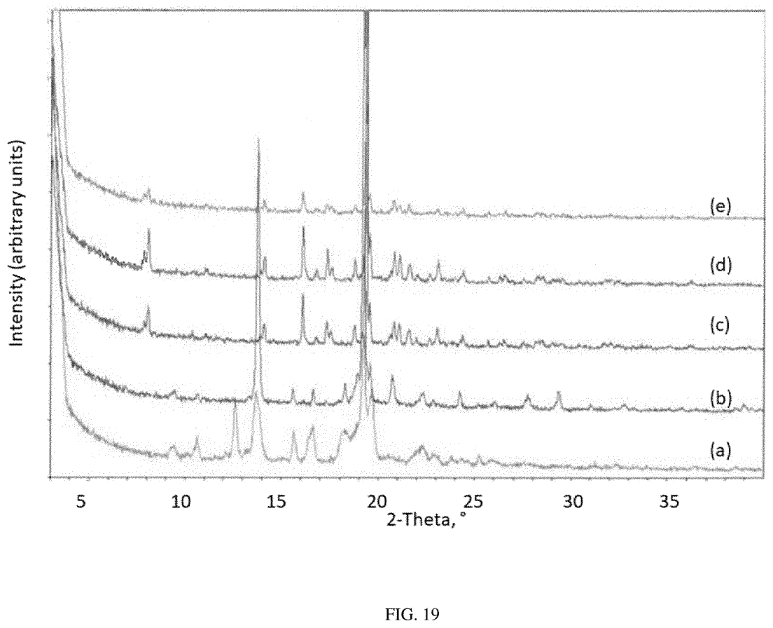

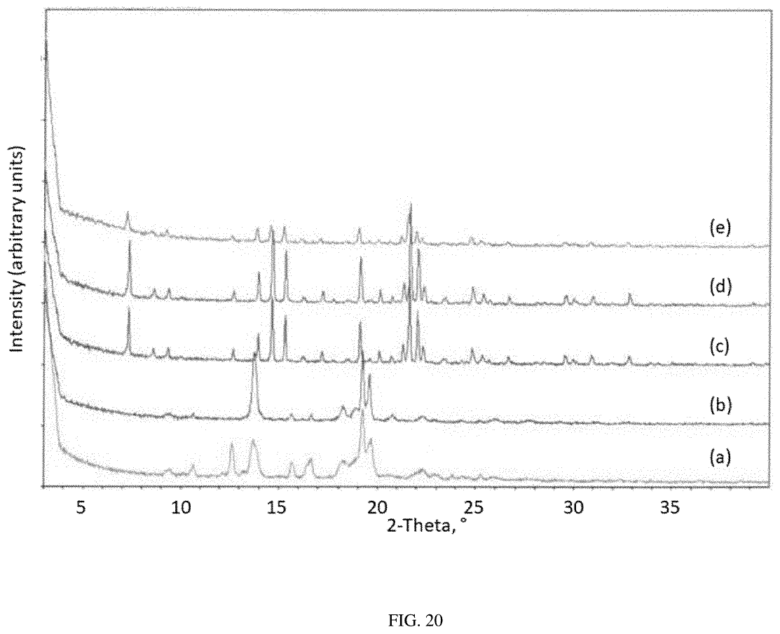

PXRD for the salts 9, 10, and 11 are shown in FIGS. 18, 19, and 20, respectively. For comparison, each figure contains the following traces:

(a) Example 1 compound;

(b) Example 1 compound obtained from the reaction solvent (Table 36)

(c) salt as prepared from the preliminary salt formation experiment;

(d) salt as prepared for the 100 mg scale-up; and

(e) salt as prepared for the 500 mg scale-up.

TABLE 37
XRPD of hydrogen fumarate salt 11 from 500 mg scale-up reaction

| | | relative |
|---|---|---|
| 2-Theta, ° | d-spacing, Å | height |
| 3.58 | 24.66 | 100.0 |
| 7.25 | 12.19 | 29.0 |
| 8.50 | 10.39 | 6.2 |
| 9.01 | 9.81 | 5.0 |
| 9.27 | 9.53 | 11.0 |
| 12.64 | 7.00 | 8.6 |
| 13.89 | 6.37 | 19.9 |
| 14.60 | 6.06 | 27.7 |
| 15.27 | 5.80 | 26.1 |
| 16.21 | 5.46 | 5.9 |
| 17.14 | 5.17 | 7.9 |
| 19.04 | 4.66 | 26.9 |
| 19.58 | 4.53 | 4.1 |
| 20.03 | 4.43 | 6.7 |
| 20.64 | 4.30 | 4.1 |
| 21.24 | 4.18 | 12.4 |
| 21.60 | 4.11 | 50.3 |
| 21.99 | 4.04 | 21.3 |
| 24.78 | 3.59 | 12.9 |
| 25.29 | 3.52 | 6.7 |
| 26.63 | 3.35 | 6.4 |
| 29.55 | 3.02 | 7.0 |
| 29.96 | 2.98 | 4.1 |
| 30.85 | 2.90 | 6.8 |
| 32.78 | 2.73 | 6.5 |

### Scale-Up (1000 mg) for Hydrogen Fumarate Salt of the Example 1 Compound

About 1000 mg of the Example 1 compound and 291.88 mg of fumaric acid (1.1 equiv) were combined with 25 mL of acetone, and the mixture was stirred at 50° C. (200 RPM) for 4 hr. The mixture was cooled to room temperature overnight. The solid precipitate that had formed was removed by centrifugation and dried under vacuum.

Solubility of the salt formed in the 1000 mg scale-up batch is reported in Table 38, below.

TABLE 38
Solubility of 11.

| | Solubility | | | Solubility | |
|---|---|---|---|---|---|
| Solvent | RT | 50° C. | Solvent | RT | 50° C. |
| MeOH | 25-50 | 50-100 | DMF | >100 | N/A |
| EtOH | <20 | 20-33 | DMSO | <100 | >100 |
| IPA | 10-20 | 20-33 | DCM | <10 | <10 |
| ACN | <10 | <10 | Toluene | <10 | <10 |
| Acetone | <10 | 10-20 | Heptane | <10 | <10 |
| MEK | <10 | 10-20 | H₂O | <10 | 10-20 |
| MTBE | <10 | <10 | Dioxane | <20 | 20-33 |
| EtOAc | <10 | <10 | 1/1 (v/v) EtOH/ | 20-33 | 50-100 |
| | | | H₂O | | |
| THF | <10 | 33-50 | 1/1 (v/v) Acetone/ | <33 | 50-100 |
| | | | H₂O | | |

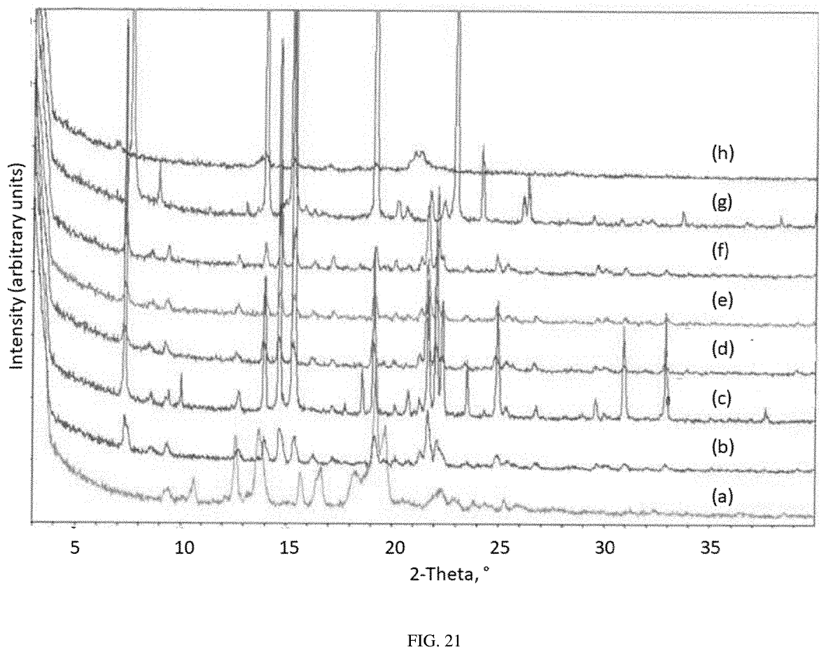

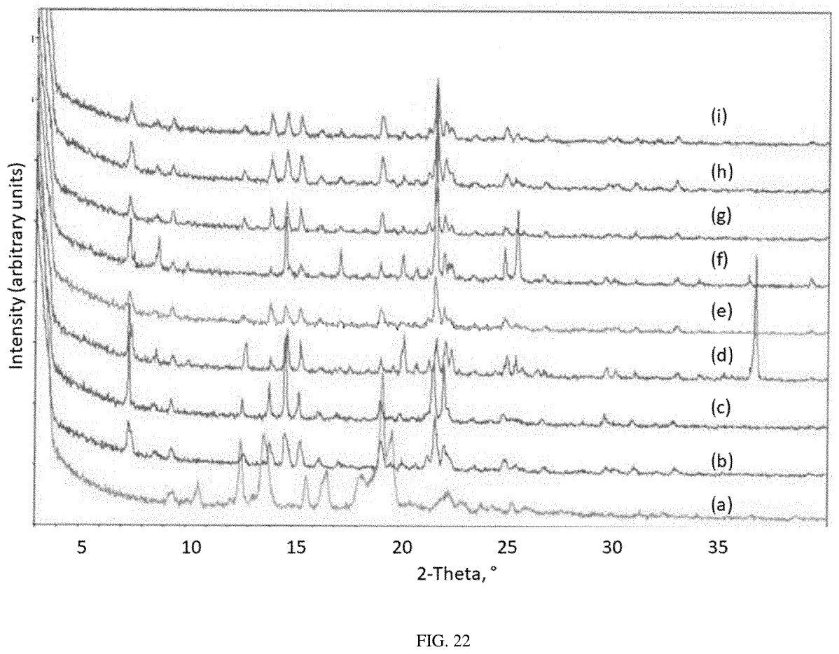

Formation of polymorphs of the hydrogen fumarate salt 11 was examined. About 15 mg of hydrogen fumarate salt 11 was combined with 0.5 mL of a selected organic solvent in a vial. The mixture was stirred at 700 RPM at 50° C. for 2 days. If a solid precipitate was present, the solid precipitate was then removed by centrifugation (14,000 RPM, 5 min) and dried under vacuum at 30° C. (Method I). If a solid precipitate was not present, the solvent was removed by evaporation under vacuum at 30° C. (Method II). In either case, the resulting material was evaluated by XRPD. Diffractograms are provided in FIGS. 21 and 22. For both figures, trace (a) is the Example 1 compound, and trace (b) is the product from the 1000 mg scale-up batch; the remainder of the traces represent polymorph formation experiments.

TABLE 39
Polymorphism of the hydrogen fumarate salt 11.

| | Observation | | Dry | | | XRPD |
|---|---|---|---|---|---|---|
| Solvent | rt | 50° C. | method | Appearance | FIGS. | pattern |
| Acetone | | c | II | white powder | 21(c) | A |
| CH₃CN | | s | I | white powder | 21(d) | A |
| EtOAc | | s | I | white powder | 21(e) | A |
| EtOH | | c | II | white powder | 21(f) | A |
| iPrOH | | c | II | white powder | 21(g) | B |
| MeOH | | c | II | pale yellow | 21(h) | almost |
| | | | | powder | | amor- |
| | | | | | | phous |
| Dioxane | | c | II | pale yellow | 22(c) | A |
| | | | | powder | | |
| 1/1 (v/v) | | c | II | pale yellow | 22(d) | A |
| acetone/H₂O | | | | powder | | |
| CH₂Cl₂ | | s | I | white powder | 22(e) | A |
| 1/1 (v/v) | | c | II | pale yellow | 22(f) | A |
| EtOH/H₂O | | | | powder | | |
| H₂O | | s | I | pale yellow | 22(g) | A |
| | | | | powder | | |
| Heptane | | s | I | white powder | 22(h) | A |
| Toluene | | s | I | white powder | 22(i) | A |

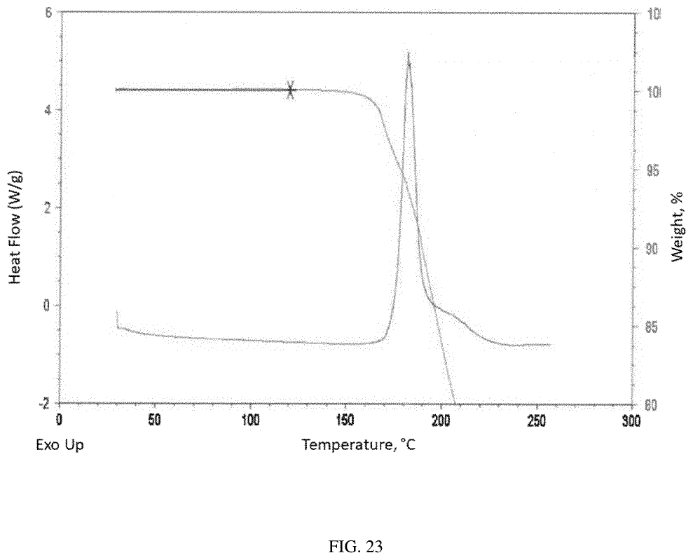

DSC and TGA for the hydrogen fumarate salt 11 from acetone are presented in FIG. 23. The sample showed 0.067% weight loss up to 119.92° C.

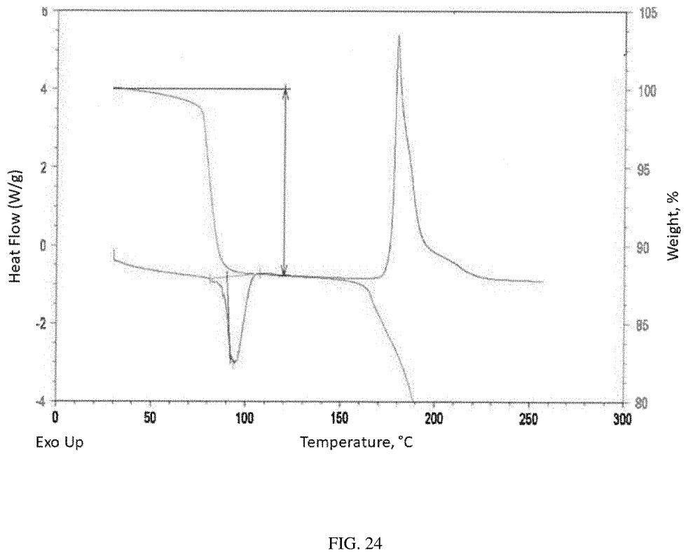

DSC and TGA for the hydrogen fumarate salt 11 from iPrOH are presented in FIG. 24. The sample showed 11.93% weight loss up to 119.92° C.

TABLE 40
XRPD of hydrogen fumarate salt 11 form “B” from iPrOH

| | | relative |
|---|---|---|
| 2-Theta, ° | d-spacing, Å | height |
| 3.21 | 27.54 | 6.8 |
| 3.66 | 24.13 | 2.9 |
| 7.65 | 11.54 | 100.0 |
| 8.37 | 10.56 | 0.3 |
| 8.94 | 9.88 | 1.6 |
| 11.33 | 7.80 | 0.3 |
| 13.11 | 6.75 | 0.5 |
| 13.63 | 6.49 | 0.3 |
| 14.03 | 6.31 | 16.6 |
| 14.82 | 5.97 | 0.4 |
| 14.95 | 5.92 | 0.6 |
| 15.29 | 5.79 | 48.4 |
| 15.87 | 5.58 | 0.3 |
| 19.19 | 4.62 | 17.7 |
| 20.22 | 4.39 | 0.7 |
| 20.65 | 4.30 | 0.5 |
| 21.78 | 4.08 | 1.2 |
| 22.46 | 3.95 | 0.8 |
| 22.98 | 3.87 | 26.3 |
| 24.19 | 3.68 | 3.1 |
| 26.12 | 3.41 | 1.1 |
| 26.35 | 3.38 | 2.0 |
| 29.43 | 3.03 | 0.4 |
| 33.67 | 2.66 | 0.7 |
| 38.31 | 2.35 | 0.5 |

### Scale-Up (1500 mg) of Hydrogen Fumarate Salt 11

About 1.5 g of the Example 1 compound and 437.82 mg of fumaric acid (1.1 equiv) were combined with 37.5 mL of acetone, and the mixture was stirred at 50° C. (200 RPM) for 4 hr. The mixture was cooled to room temperature overnight. The solid precipitate that had formed was removed by centrifugation and dried under vacuum.

### Polymorph Formation Experiments of Hydrogen Fumarate Salt 11

Polymorph formation was examined with a slurry method using various solvents. About 500 mg of hydrogen fumarate salt 11 was combined with 50 mL of DMSO and 1 mL DMF to afford a saturated solution. Anti-solvent (no more than 5x solution volume) chosen from EtOH, iPrOH, acetone, water, and EtOAc was added. In no case was a precipitate formed.

Polymorph formation was further examined with a heat-cool method using various solvents. About 100 mg of hydrogen fumarate salt 11 was combined with 1 mL of a selected solvent. The mixture was stirred at 700 RPM for 4 hrs at 60° C., then allowed to cool to room temperature. The cycle was repeated twice. If a solid precipitate was present, the solid precipitate was then removed by centrifugation and dried under vacuum at 30° C. (Method I). If a solid precipitate was not present, the solvent was removed by evaporation (Method II). In either case, the resulting material was evaluated by XRPD.

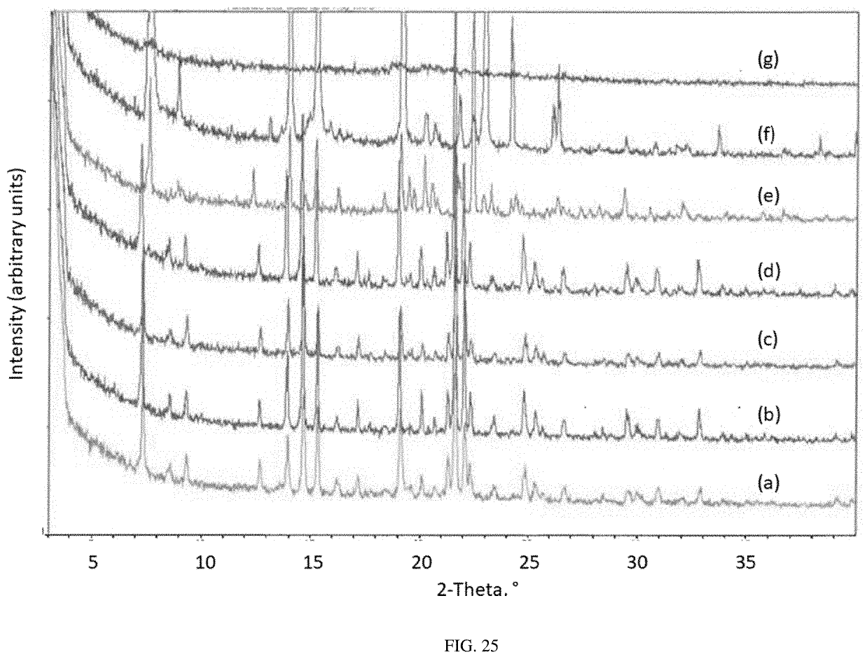

XRPD diffractograms from these experiments are displayed in FIG. 25. Trace (a) shows the hydrogen fumarate salt 11 from 1500 mg scale-up. Traces (b), (c), (d), and (e) show the solid precipitate from the heat-cool experiment using EtOAc, EtOH, acetone, and iPrOH, respectively. For comparison, trace (f) shows the evaporation product from the slurry method using iPrOH. Trace (g) shows the evaporation product from THF.

TABLE 41
Polymorphism of hydrogen fumarate salt 11 from various organic solvents
“s” = slurry; “c” = clear

| | observation | | Dry | | |
|---|---|---|---|---|---|
| Solvent | rt | 60° C. | method | appearance | XRPD results |
| EtOAc | s | s | I | white powder | A |
| EtOH | s | s | I | white powder | A |
| Acetone | s | s | I | white powder | A |
| iPrOH | s | s | I | white powder | similar to B |
| THF | c | c | II | white powder | amorphous |

### Competitive Slurry of Hydrogen Fumarate Salt 11 Polymorphs

The pattern A polymorph was obtained from acetone in the previous study. The pattern B polymorph was obtained from iPrOH in the previous study. A suspension of the indicated polymorph(s) was stirred in acetone at 500 RPM for 3 days at 50° C. The residue was removed by centrifugation, and dried 4 hr under vacuum at 30° C.

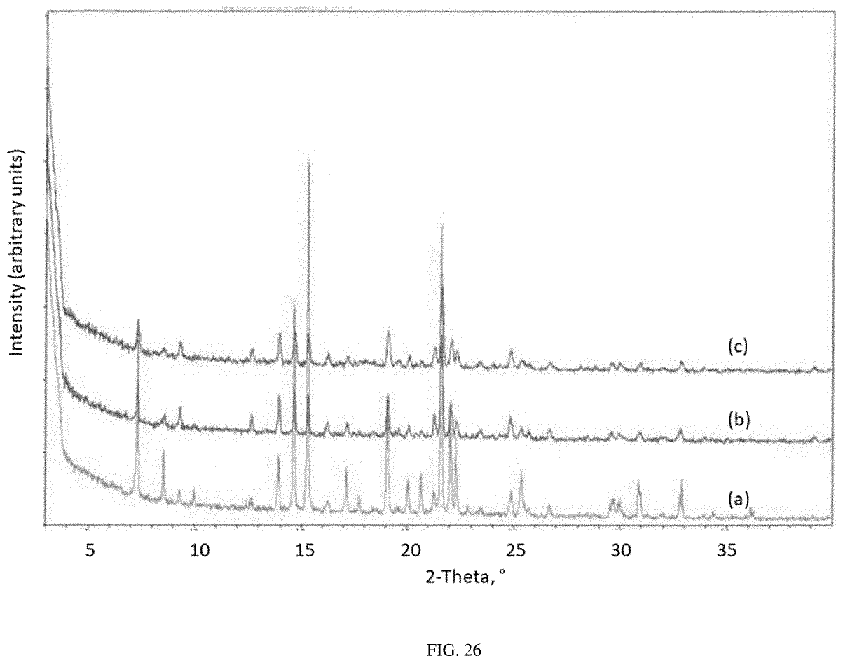

XRPD diffractograms from these experiments are shown in FIG. 26. Traces (a) and (b) show the products that were formed upon suspension of the pattern A polymorph and pattern B polymorph, respectively. Trace (c) shows the product that was formed upon suspension of a mixture of the pattern A polymorph and the pattern B polymorph.

TABLE 42
Competitive slurry experiment.

| | | solvent | | |
|---|---|---|---|---|
| mass A | mass B | volume | appearance | XRPD |
| 30.0 | — | 1.0 | pale yellow powder | A |
| 30.0 | 30.0 | 2.0 | white powder | A |
| — | 30.0 | 1.0 | white powder | A |

### Scale-Up (8500 mg) of Hydrogen Fumarate Salt 11

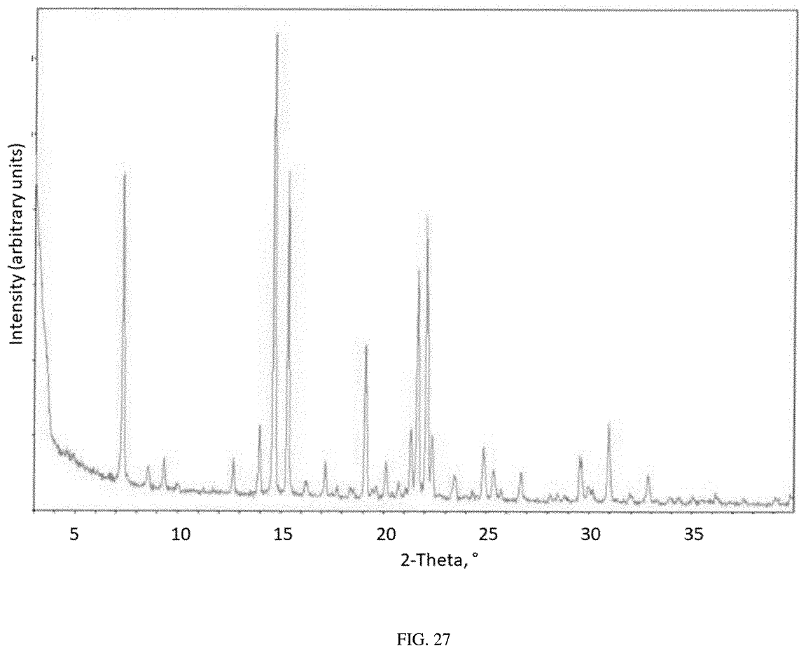

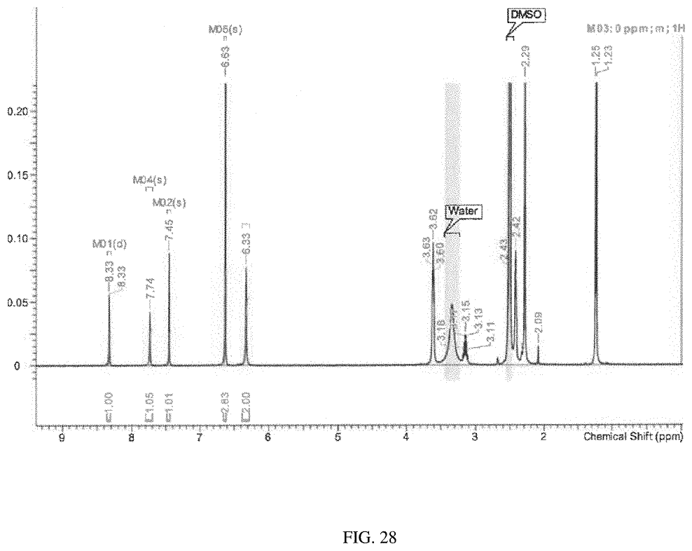

About 8.5 g of the Example 1 compound and 2480.98 mg of fumaric acid (1.1 equiv) were combined with 212.5 mL of acetone, and the mixture was stirred at 50° C. (600 RPM) for 4 hr. The mixture was cooled to room temperature overnight. The solid precipitate that had formed was removed by filtration and dried under vacuum. FIG. 27 shows the hydrogen fumarate salt 11 from 1500 mg scale-up. FIG. 28 shows the ¹H NMR spectrum (DMSO-d₆) of the hydrogen fumarate salt 11 from 1500 mg scale-up.

TABLE 43
Summary of Example 1 compound and scale-up hydrogen
fumarate salt 11.

| | | hydrogen fumarate salt |
|---|---|---|
| | Example 1 compound | 11 |
| Color | white powder | off-white powder |
| Purity | 99.91% | 100% |
| Salt ratio (¹H-NMR) | N/A | 1.50:1 |
| Yield | N/A | 64.44% |
| Crystallinity (XRPD) | medium | high |
| mp, ° C. | 240.33 | no mp |
| enthalpy (DSC, J/g) | 71.67 | N/A |
| weight loss (TGA, %) | 0.29% (<120° C.) | 0.11% (<120° C.) |
| PSD | N/A | D₉₀ = 43 uM |
| molecular weight | 437.46 | 437.46 |
| formula weight | 437.46 | 553.53 |
| formula | C₂₁H₂₆F₃N₅O₂ | C₂₁H₂₆F₃N₅O₂•C₄H₄O₄ |
| amount | N/A | ~7 g |

### Spectral Data for the Hydrogen Fumarate Salt 11

¹H NMR in DMSO-d₆ was observed at 25.00° C. Data collection parameters: 600.13 MHz; 16 transients; receiver gain 575.00; sweep width 12375.86 Hz; spectrum offset 3700.3259 Hz; referenced against TMS.

δ 13.13 (br s, 3H), 8.32 (s, 1H), 7.73 (s, 1H), 7.44 (s, 1H), 6.63 (s, 3H), 6.30 (s, 2H), 3.62-3.60 (m, 4H), 3.17-3.13 (m, 1H), 2.42 (appar br s, 4H), 2.29 (s, 6H), 1.23 (d, J=6.8 Hz, 6H).

¹³C NMR in DMSO-d₆ was observed at 25.15° C. Data collection parameters: 125.76 MHz; 1024 transients; receiver gain 191.79; sweep width 29761.00 Hz; spectrum offset 12502.6699 Hz; referenced against TMS.

| # | ppm | Hz | height |
|---|---|---|---|
| | 0.00 | 0 | 0.0070 |
| 1 | 22.01 | 2767.5 | 0.0398 |
| 2 | 25.75 | 3238.0 | 0.0107 |
| 3 | 46.32 | 5825.6 | 0.0152 |
| 4 | 48.34 | 6079.0 | 0.0335 |
| 5 | 50.72 | 6378.7 | 0.0411 |
| 6 | 54.81 | 6892.8 | 0.0104 |
| 7 | 65.64 | 8255.2 | 0.0362 |
| 8 | 114.00 | 14336.9 | 0.0118 |
| 9 | 119.46 | 15023.5 | 0.0022 |
| 10 | 119.88 | 15075.3 | 0.0047 |
| 11 | 121.51 | 15280.6 | 0.0025 |
| 12 | 124.64 | 15673.9 | 0.0087 |
| 13 | 130.08 | 16358.7 | 0.0054 |
| 14 | 133.88 | 16836.4 | 0.0566 |
| 15 | 134.67 | 16936.3 | 0.0077 |
| 16 | 142.42 | 17910.0 | 0.0104 |
| 17 | 151.05 | 18996.3 | 0.0097 |
| 18 | 153.00 | 19241.5 | 0.0078 |
| 19 | 165.87 | 20859.1 | 0.0311 |

### Particle Size Distribution for the Hydrogen Fumarate Salt 11

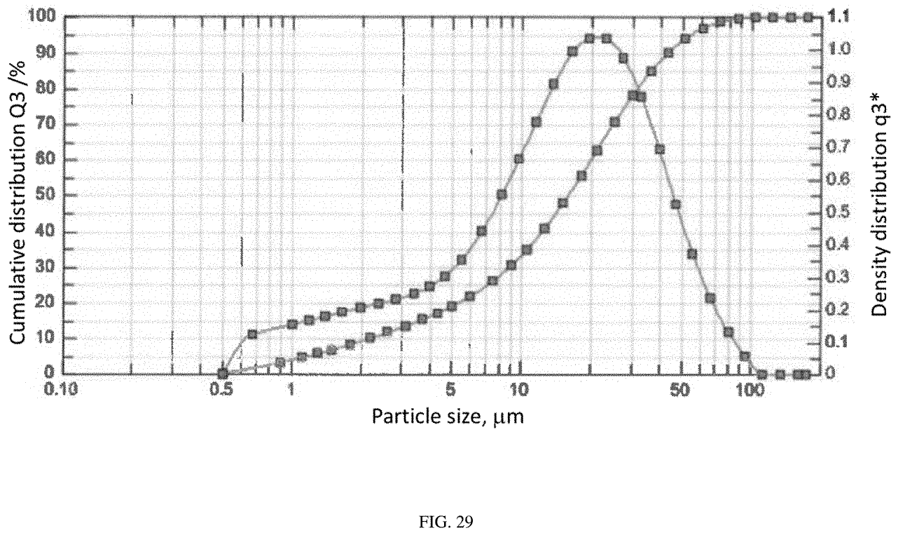

Particle size distribution of hydrogen fumarate salt 11 is shown in FIG. 29. Distributions at various levels are presented in Table 44. VMD=20.02 μM; STD=17.09 μM; Duration=0.04; Copt=2.90.

TABLE 44
Particle size distribution for the hydrogen fumarate salt 11 at
various levels.

| % level | D-value |
|---|---|
| 5 | 1.20 |
| 10 | 2.21 |
| 15 | 3.64 |
| 20 | 5.39 |
| 25 | 7.16 |
| 30 | 8.89 |
| 35 | 10.58 |
| 40 | 12.28 |
| 45 | 14.04 |
| 50 | 15.88 |
| 55 | 17.79 |
| 60 | 19.93 |
| 65 | 22.30 |
| 70 | 24.85 |
| 75 | 28.06 |
| 80 | 31.79 |
| 85 | 36.34 |
| 90 | 42.91 |
| 95 | 54.73 |

The activity of the Example 1 compound as a DLK inhibitor is illustrated in the following assays. It is expected that the salts disclosed herein will have similar activities as DLK inhibitors.

### Biological Activity Assays

Compounds described herein have been shown to bind DLK in vitro, and to inhibit phosphorylation of a downstream molecular target in a cellular assay.

### DLK K_{d} Determinations

The DLK dissociation constants (K_{d}) have been determined in the KINOMEscan KdELECT Service at DiscoveRx.

A fusion protein of full length of human DLK (amino acids 1-859) and the DNA binding domain of NFkB was expressed in transiently transfected HEK293 cells. From these HEK 293 cells, extracts were prepared in M-PER extraction buffer (Pierce) in the presence of Protease Inhibitor Cocktail Complete (Roche) and Phosphatase Inhibitor Cocktail Set II (Merck) per manufacturers' instructions. The DLK fusion protein was labeled with a chimeric double-stranded DNA tag containing the NFkB binding site (5′-GGGAATTCCC-3′) fused to an amplicon for qPCR readout, which was added directly to the expression extract (the final concentration of DNA-tag in the binding reaction is 0.1 nM).

Streptavidin-coated magnetic beads (Dynal M280) were treated with a biotinylated small molecule ligand for 30 minutes at room temperature to generate affinity resins the binding assays. The liganded beads were blocked with excess biotin and washed with blocking buffer (SeaBlock (Pierce), 1% BSA, 0.05% Tween 20, 1 mM DTT) to remove unbound ligand and to reduce nonspecific binding.

The binding reaction was assembled by combining 16 al of DNA-tagged kinase extract, 3.8 al liganded affinity beads, and 0.18 al test compound (PBS/0.05% Tween 20/10 mM DTT/0.1% BSA/2 ag/ml sonicated salmon sperm DNA)]. Extracts were used directly in binding assays without any enzyme purification steps at a ≥10,000-fold overall stock dilution (final DNA-tagged enzyme concentration <0.1 nM). Extracts were loaded with DNA-tag and diluted into the binding reaction in a two step process. First extracts were diluted 1:100 in 1x binding buffer (PBS/0.05% Tween 20/10 mM DTT/0.1% BSA/2 μg/ml sonicated salmon sperm DNA) containing 10 nM DNA-tag. This dilution was allowed to equilibrate at room temperature for 15 minutes and then subsequently diluted 1:100 in 1× binding buffer. Test compounds were prepared as 111× stocks in 100% DMSO. K_{d}s were determined using an 11-point 3-fold compound dilution series with three DMSO control points. All compounds for K_{d} measurements are distributed by acoustic transfer (non-contact dispensing) in 100% DMSO. The compounds were then diluted directly into the assays such that the final concentration of DMSO was 0.9%. All reactions performed in polypropylene 384-well plates. Each was a final volume of 0.02 mL. Assays were incubated with shaking for 1 hour at room temperature. Then the beads were pelleted and washed with wash buffer (lx PBS, 0.05% Tween 20) to remove displaced kinase and test compound. The washed based were re-suspended in elution buffer (lx PBS, 0.05% Tween 20, 0.5 μM non-biotinylated affinity ligand) and incubated at room temperature with shaking for 30 minutes. The kinase concentration in the eluates was measured by qPCR. qPCR reactions were assembled by adding 2.5 μL of kinase eluate to 7.5 μL of qPCR master mix containing 0.15 μM amplicon primers and 0.15 μM amplicon probe. The qPCR protocol consisted of a 10 minute hot start at 95° C., followed by 35 cycles of 95° C. for 15 seconds, 60° C. for 1 minute.

Test Compound Handling.

Test compounds were prepared as 111× stocks in 100% DMSO. K_{d}s were determined using an 11-point 3-fold compound dilution series with three DMSO control points. All compounds for K_{d} measurements are distributed by acoustic transfer (non-contact dispensing) in 100% DMSO. The compounds were then diluted directly into the assays such that the final concentration of DMSO was 0.9%. The K_{d}s were determined using a compound top concentration of 30,000 nM. K_{d} measurements were performed in duplicate.

Binding Constant (K_{d}) Calculation.

Binding constants (K_{d}s) were calculated with a standard dose-response curve using the Hill equation:

$$Response = Background + \frac{( Signal - Background )}{( 1 + ( \frac{{Kd}^{Hill Slope}}{{Dose}^{Hill Slope}} )}$$

The Hill Slope was set to −1. Curves were fitted using a non-linear least square fit with the Levenberg-Marquardt algorithm (Levenberg, K., A method for the solution of certain non-linear problems in least squares, Q. Appl. Math. 2, 164-168 (1944)). See also Fabian, M. A. et al. A small molecule-kinase interaction map for clinical kinase inhibitors. Nat. Biotechnol. 23, 329-336 (2005); Wodicka, L. M. et al. Activation state-dependent binding of small molecule kinase inhibitors: structural insights from biochemistry. Chem Biol. 17, 1241-9 (2010).

Compounds with lower dissociation constants bind with more affinity to the target. Compounds disclosed herein, particularly (but not exclusively) those with lower dissociation constants, can be expected to inhibit target activity and to be useful in the treatment of DLK-mediated disease. Results are reported below in Table 45 in nM.

### Phospho-cJun Cellular Assay

HEK293 cells stably transfected with a Dox-inducible human DLK were plated into a 384-well plate in 20 μl (40,000 cells/well) of DMEM medium (without phenol red) containing 10% fetal bovine serum, 1.5 μg/ml doxycycline and 1 μg/ml puromycin. The cells as negative control were grown in the absence of doxycycline. The plate was incubated at 37° C., 5% CO₂ for 20 h, before DMSO (control) or compounds diluted in medium were added. The cells were incubated at 37° C. for an additional 5 h, followed by lysis and the addition detection antibodies from p-cJun (Ser63) cellular assay kit (Cisbio) per manufacturer protocol. The standard dose response curves were fitted by Genedata Screener software using the variable-slope model: Signal=Signal_{negative control}+(Signal_{DMSO control}−Signal_{negative control})/(1+(IC₅₀/Dose)\hat{ }Hill slope). Only signal and dose in the equation were treated as known values. Results are reported below in Table 45 in nM.

TABLE 45
DLK activity

| | | Cell |
|---|---|---|
| Ex. | Kd, nM | IC50 |
| 1 | 11 | 367.8 |

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this disclosure, and without departing from the spirit and scope thereof, can make various changes and modifications of the disclosure to adapt it to various usages and conditions.

What is claimed is:

1. A compound of structural Formula I 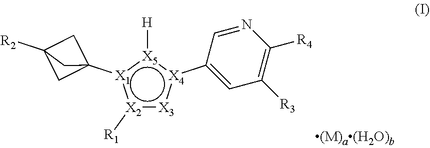
or a polymorph thereof, wherein:
X₁ is selected from C and N;
X₂ is selected from C and N;
exactly one of X₁ and X₂ is N;
X₃ is N;
X₄ and X₅ are C;
X₁, X₂, X₃, X₄, and X₅ form a five membered heteroaryl;
R₁ is selected from alkyl, cycloalkyl, and heterocycloalkyl, any of which is optionally substituted with one to three R₅ groups;
R₂ is H or is selected from alkyl, amino, aryl, cycloalkyl, haloalkyl, heteroalkyl, heteroaryl, heterocycloalkyl, and sulfonylalkyl, any of which is optionally substituted with one to three R₆ groups;
R₃ is selected from H, alkyl, (alkoxy)alkyl, (arylalkoxy)alkyl, (heteroarylalkoxy)alkyl, cyano, cycloalkyl, halo, haloalkoxy, and haloalkyl;
R₄ is N(R₄ₐ)₂, wherein each R₄ₐ is independently selected from hydrogen, C₁₋₄alkyl, and C₁₋₄haloalkyl;
or R₃ and R₄ together with the atoms to which they are attached form a 5- or 6-membered heteroaryl or heteroalkyl ring, optionally substituted with one to three R₇ groups;
each R₅ and R₆ is independently selected from C₁₋₄alkyl, C₁₋₄haloalkyl, C₁₋₄alkoxy, C₁₋₄haloalkoxy, C₁₋₄alkylthio, C₁₋₄haloalkylthio, aryl, heteroaryl, C₃₋₇cycloalkyl, C₃₋₇heterocycloalkyl, (aryl)C₁₋₄alkyl, (heteroaryl)C₁₋₄alkyl, (C₃₋₇cycloalkyl)C₁₋₄alkyl, (C₃₋₇heterocycloalkyl)C₁₋₄alkyl, (ethenyl)C₁₋₄alkyl, (ethynyl)C₁₋₄alkyl, (aryl)C₁₋₄alkoxy, (heteroaryl)C₁₋₄alkoxy, (C₃₋₇cycloalkyl)C₁₋₄alkoxy, (C₃₋₇heterocycloalkyl)C₁₋₄alkoxy, (aryl)C₁₋₄alkylthio, (heteroaryl)C₁₋₄alkylthio, (C₃₋₇cycloalkyl)C₁₋₄alkylthio, (C₃₋₇heterocycloalkyl)C₁-4alkylthio, amino, halo, hydroxy, cyano, and oxo;
each R₇ is independently selected from C₁₋₄alkyl, C₁₋₄haloalkyl, C₁₋₄alkoxy, C₁₋₄haloalkoxy, aryl, heteroaryl, C₃₋₇cycloalkyl, C₃₋₇heterocycloalkyl, (aryl)C₁₋₄alkyl, (heteroaryl)C₁₋₄alkyl, (C₃₋₇cycloalkyl)C₁₋₄alkyl, (C₃₋₇heterocycloalkyl)C₁₋₄alkyl, halo, hydroxy, cyano, and oxo;
a is a fractional or whole number between about 0.5 and 1.5, inclusive;
b is a fractional or whole number between about 0 and 10, inclusive; and
M is selected from hydrochloric acid, sulfuric acid, phosphoric acid, maleic acid, fumaric acid, tartaric acid, succinic acid, L-malic acid, citric acid, and methanesulfonic acid.

2. The compound of claim 1, wherein R₁ is isopropyl.

3. The compound of either one of claims 1 and 2, wherein R₂ is morpholin-1-yl.

4. The compound of any one of claims 1 through 3, wherein R₃ is selected from difluoromethoxy, trifluoromethoxy, and trifluoromethyl.

5. The compound of claim 4, wherein R₃ is trifluoromethoxy.

6. The compound of any one of claims 1 through 5, wherein R₄ is NH₂.

7. The compound of claim 1, having structural Formula II: 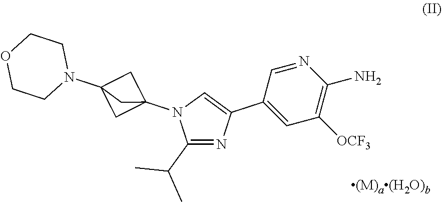 [SMILES: CC(C)c1nc(-c2cnc(N)c(OC(F)(F)F)c2)cn1C12CC(N3CCOCC3)(C1)C2]
or a polymorph thereof, wherein:
a is a fractional or whole number between about 0.5 and 1.5, inclusive;
b is a fractional or whole number between about 0 and 10, inclusive; and
M is selected from hydrochloric acid, sulfuric acid, phosphoric acid, maleic acid, fumaric acid, tartaric acid, succinic acid, L-malic acid, citric acid, and methanesulfonic acid.

8. The compound as recited in any one of claims 1 through 7, wherein the compound is in a solid form.

9. The compound as recited in claim 8, wherein the compound is in a crystalline form.

10. The compound as recited in any one of claims 1 through 9, wherein a is 1.5.

11. The compound as recited in claim 10, wherein M is chosen from tartaric acid, L-malic acid, citric acid, maleic acid, succinic acid, and fumaric acid.

12. The compound as recited in claim 11, wherein M is chosen from citric acid, maleic acid, succinic acid, and fumaric acid.

13. The compound as recited in claim 12, wherein M is chosen from maleic acid, succinic acid, and fumaric acid.

14. The compound as recited in claim 13, wherein M is fumaric acid.

15. The compound as recited in claim 9, chosen from:
5-(2-isopropyl-1-(3-morpholinobicyclo[1.1.1]pentan-1-yl)-1H-imidazol-4-yl)-3-(trifluoromethoxy)pyridin-2-amine hydrogen tartrate;
5-(2-isopropyl-1-(3-morpholinobicyclo[1.1.1]pentan-1-yl)-1H-imidazol-4-yl)-3-(trifluoromethoxy)pyridin-2-amine hydrogen L-malate;
5-(2-isopropyl-1-(3-morpholinobicyclo[1.1.1]pentan-1-yl)-1H-imidazol-4-yl)-3-(trifluoromethoxy)pyridin-2-amine dihydrogen citrate;
5-(2-isopropyl-1-(3-morpholinobicyclo[1.1.1]pentan-1-yl)-1H-imidazol-4-yl)-3-(trifluoromethoxy)pyridin-2-amine hydrogen maleate;
5-(2-isopropyl-1-(3-morpholinobicyclo[1.1.1]pentan-1-yl)-1H-imidazol-4-yl)-3-(trifluoromethoxy)pyridin-2-amine hydrogen succinate; and
5-(2-isopropyl-1-(3-morpholinobicyclo[1.1.1]pentan-1-yl)-1H-imidazol-4-yl)-3-(trifluoromethoxy)pyridin-2-amine hydrogen fumarate.

16. The compound as recited in claim 15, chosen from:
5-(2-isopropyl-1-(3-morpholinobicyclo[1.1.1]pentan-1-yl)-1H-imidazol-4-yl)-3-(trifluoromethoxy)pyridin-2-amine dihydrogen citrate;
5-(2-isopropyl-1-(3-morpholinobicyclo[1.1.1]pentan-1-yl)-1H-imidazol-4-yl)-3-(trifluoromethoxy)pyridin-2-amine hydrogen maleate;
5-(2-isopropyl-1-(3-morpholinobicyclo[1.1.1]pentan-1-yl)-1H-imidazol-4-yl)-3-(trifluoromethoxy)pyridin-2-amine hydrogen succinate; and
5-(2-isopropyl-1-(3-morpholinobicyclo[1.1.1]pentan-1-yl)-1H-imidazol-4-yl)-3-(trifluoromethoxy)pyridin-2-amine hydrogen fumarate.

17. The compound as recited in claim 16, chosen from:
5-(2-isopropyl-1-(3-morpholinobicyclo[1.1.1]pentan-1-yl)-1H-imidazol-4-yl)-3-(trifluoromethoxy)pyridin-2-amine hydrogen maleate;
5-(2-isopropyl-1-(3-morpholinobicyclo[1.1.1]pentan-1-yl)-1H-imidazol-4-yl)-3-(trifluoromethoxy)pyridin-2-amine hydrogen succinate; and
5-(2-isopropyl-1-(3-morpholinobicyclo[1.1.1]pentan-1-yl)-1H-imidazol-4-yl)-3-(trifluoromethoxy)pyridin-2-amine hydrogen fumarate.

18. The compound as recited in claim 16, wherein the compound is 5-(2-isopropyl-1-(3-morpholinobicyclo[1.1.1]pentan-1-yl)-1H-imidazol-4-yl)-3-(trifluoromethoxy)pyridin-2-amine dihydrogen citrate.

19. The compound as recited in claim 18, characterized by the presence of XRPD peaks with d-spacings of about 26.87, 24.26, 6.62, 5.86, 5.15, 4.68, 4.60, 4.52, 4.38, and 4.19 Å.

20. The compound as recited in claim 16, wherein the compound is 5-(2-isopropyl-1-(3-morpholinobicyclo[1.1.1]pentan-1-yl)-1H-imidazol-4-yl)-3-(trifluoromethoxy)pyridin-2-amine hydrogen maleate.

21. The compound as recited in claim 20, characterized by the presence of XRPD peaks with d-spacings of about 24.26, 7.77, 5.52, 4.76, 4.52, 4.36, 4.27, 4.09, 3.97, and 3.91 Å.

22. The compound as recited in claim 16, wherein the compound is 5-(2-isopropyl-1-(3-morpholinobicyclo[1.1.1]pentan-1-yl)-1H-imidazol-4-yl)-3-(trifluoromethoxy)pyridin-2-amine hydrogen succinate.

23. The compound as recited in claim 22, characterized by the presence of XRPD peaks with d-spacings of about 4.03, 3.79, 3.24, 3.07, 3.05, 3.02, 2.94, 2.81, 2.79, and 2.75 Å.

24. The compound as recited in claim 16, wherein the compound is 5-(2-isopropyl-1-(3-morpholinobicyclo[1.1.1]pentan-1-yl)-1H-imidazol-4-yl)-3-(trifluoromethoxy)pyridin-2-amine hydrogen fumarate.

25. The compound as recited in claim 24, characterized by the presence of XRPD peaks with d-spacings of about 26.87, 24.26, 12.00, 6.33, 6.02, 5.76, 4.63, 4.16, 4.10, and 4.02 Å.

26. A compound having structural Formula III: 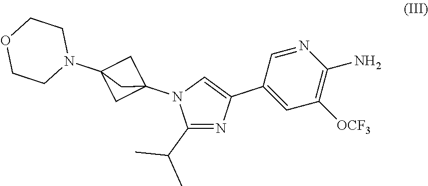 [SMILES: CC(C)c1nc(-c2cnc(N)c(OC(F)(F)F)c2)cn1C12CC(N3CCOCC3)(C1)C2]
wherein the compound is Polymorph A.

27. A compound as recited in claim 1 for use as a medicament.

28. A compound as recited in claim 1 for use in the treatment of a disease mediated by DLK.

29. A compound as recited in claim 1 for use in the manufacture of a medicament for the prevention or treatment of a disease ameliorated by the inhibition of DLK.

30. A pharmaceutical composition comprising a compound as recited in claim 1 together with a pharmaceutically acceptable carrier.

31. A method of inhibition of DLK comprising contacting DLK with a compound as recited in any preceding claim.

32. A method of treatment of a DLK-mediated disease comprising the administration of a therapeutically effective amount of a compound as recited in any preceding claim to a patient in need thereof.

33. The method as recited in claim 32 wherein said disease is a neurological disease.

34. The method as recited in claim 33, wherein said neurological disease results from traumatic injury to central nervous system or peripheral nervous system neurons.

35. The method as recited in claim 34, wherein said traumatic injury is chosen from stroke, traumatic brain injury, and spinal cord injury.

36. The method as recited in claim 33, wherein said neurological disease results from a chronic neurodegenerative condition.

37. The method as recited in claim 36, wherein said chronic neurodegenerative condition is chosen from Alzheimer's disease, frontotemporal dementia, Parkinson's disease, Huntington's disease, amyotrophic lateral sclerosis, spinocerebellar ataxia, progressive supranuclear palsy, Lewy body disease, and Kennedy's disease.

38. The method as recited in claim 33, wherein said neurological disease results from a neuropathy resulting from neurological damage.

39. The method as recited in claim 38, wherein said neurological damage is chosen from chemotherapy-induced peripheral neuropathy and diabetic neuropathy.

40. The method as recited in claim 32 wherein said disease is a cognitive disorder.

41. The method as recited in claim 40 wherein said cognitive disorder is caused by pharmacological intervention.

42. A method of treatment of a DLK-mediated disease comprising the administration of:
a. a therapeutically effective amount of a compound as recited in any preceding claim; and
b. another therapeutic agent.

43. The method as recited in claim 42, wherein said DLK-mediated disease is a cognitive disorder caused by pharmacological intervention.

44. The method as recited in claim 42, wherein said cognitive disorder is chemotherapy-induced cognitive disorder.

45. A method of treatment of cancer and reducing the development of a chemotherapy induced neurological disorder, comprising the administration of a chemotherapeutic drug together with a compound as recited in claim 1, or a salt thereof.

46. A method for achieving an effect in a patient comprising the administration of a therapeutically effective amount of the compound as recited in claim 1 to a patient, wherein the effect is chosen from decrease loss of neurons, reduction in cerebral atrophy, improved neurological function, improved cognition, and improved mental performance.
